(19) United States
(12) Patent Application Publication
(10) Pub. No.: US 2016/0213600 A1
(43) Pub. Date: Jul. 28, 2016

(54) SILICONE GELS FOR COSMETICS AND OTHER USES

(71) Applicant: Evonik Degussa GmbH, Essen (DE)

(72) Inventors: Michael Klostermann, Essen (DE); Susann Wiechers, Essen (DE); Jürgen Meyer, Essen (DE); Joachim Venzmer, Essen (DE); Frauke Henning, Essen (DE); Matthias Mentel, Dortmund (DE)

(73) Assignee: Evonik Degussa GmbH, Essen (DE)

(21) Appl. No.: 15/004,935

(22) Filed: Jan. 23, 2016

(30) Foreign Application Priority Data
Jan. 26, 2015 (EP) 15152458.4

(51) Int. Cl. A61K 8/895; A61Q 19/00; A61Q 19/08; A61Q 1/10; A61Q 1/04; A61Q 1/02; A61Q 17/04; A61Q 1/12; A61K 8/04; A61Q 15/00

(52) U.S. Cl. CPC A61K 8/895; A61K 8/042; A61Q 19/00; A61Q 19/08; A61Q 15/00; A61Q 1/04; A61Q 1/02; A61Q 17/04; A61Q 1/12; A61Q 19/004; A61Q 19/007; A61Q 1/10; A61K 2800/95; A61K 2800/10; A61K 2800/874; A61K 2800/596

(57) ABSTRACT

The invention provides processes for the preparation of silicone gels, and the use thereof, especially in cosmetic formulations.

### CROSS REFERENCE TO RELATED APPLICATIONS

The present application claims priority to European Application, EP 15152458.4, filed on Jan. 26, 2015, the contents of which is incorporated herein by reference in its entirety.

### FIELD OF THE INVENTION

The invention provides a process for the preparation of silicone gels, and the use thereof in particular cosmetic formulations.

### PRIOR ART

An important feature of cosmetic formulations is often that they lead to a velvety-silky skin feel following application to the skin. It is sufficiently known that these properties can be attained through the use of low molecular weight silicones, such as e.g. low-viscosity silicone oils. However, a disadvantage of this class of substance is that, as a result of their good flowability, the silky skin feel produced by them disappears relatively quickly following application. To solve this problem, in the past a series of crosslinked, polymeric silicone gel materials have been developed which are notable for a significantly longer-lasting velvety-silky skin feel. These silicone gel materials comprise crosslinked silicone polymers which have been prepared in a cosmetically acceptable solvent. The use of such crosslinked silicone gels in cosmetic formulations is described for example in the specifications U.S. Pat. No. 5,760,116 and U.S. Pat. No. 6,936,686. In the prior art, such crosslinked silicone gels are mostly prepared by hydrosilylation reactions of an Si—H-functional siloxane with an olefinically modified siloxane in the presence of a platinum catalyst.

Moreover, the specifications WO12065822, US20030049212 and US2004228821 disclose silicone gels which can be prepared from thermally crosslinkable silicones in the presence of a peroxo initiator. However, structural features of the radically polymerizable silicone macromonomers used for the preparation of these gels are not disclosed in these specifications.

The specification WO2011049896 describes the preparation and use of radically crosslinked silicone gels which have been polymerized in the presence of an organoborane initiator. Both silicone oils, such as decamethylcyclopentasiloxane, and organic oils, e.g. cosmetic esters, are listed as carrier medium. The macromonomers described in this specification are a broad, very nonspecific selection of radically polymerizable siloxanes, the influence of which on the sensory properties of the silicone gels resulting therefrom, however, is not described.

It is therefore not evident from the prior art which structural properties have to be taken into consideration when selecting a radically polymerizable silicone macromonomer in order to arrive at silicone gels with preferred haptic properties, such as e.g. a particularly velvety-silky skin feel.

Furthermore, the silicone gels described in WO2011049896 have the disadvantage that the borane initiators used as radical starters, in most cases trialkylboranes, have an acute irritancy effect on eyes and skin and can thus lead, upon exposure, to severe eye and skin damage. Short-chain alkylboranes, such as e.g. triethylborane, moreover, have acute oral toxicity. As a result of this, the use of borane-initiated silicone gels in cosmetic applications is critical. Moreover, the borates produced by solvolysis from alkylboranes have been classified as reproduction-toxic and are regulated in cosmetics [SCCS (Scientific Committee on Consumer Safety), Opinion on boron compounds, 22 Jun. 2010]. Besides health-damaging aspects, alkylboranes moreover have the disadvantage that this class of substance includes pyrophoric compounds, which hinders their handling during the preparation of the silicone gels. A further disadvantage of these borane initiators is that they often only have an adequate reactivity if they are present in the form of a borane-amine complex.

However, the use of amine compounds in cosmetic applications is likewise undesired, particularly with regard to olfactory influences of such substances.

A further disadvantage of many silicone gel materials described in the prior art is moreover that, when using an organic carrier medium, the silicone polymer often only has an inadequate compatibility to the carrier medium. This can lead to clumping of the silicone polymer in the gel ranging to macroscopic phase separation, which results in negative effects on the skin feel of corresponding materials.

Moreover, many customary silicone gel materials have the disadvantage that they are notable for very high viscosity and for poor flow properties. This is disadvantageous both during the preparation and during the processing of these materials since their handleability is greatly limited as a result of the lack of pumpability.

It was therefore an object of the invention to provide radically crosslinked silicone gels which are characterized by a long-lasting velvety skin feel and also by good processability.

### DESCRIPTION OF THE INVENTION

Surprisingly, it has been found that the object underlying the invention is achieved by silicone gels obtainable by the radical polymerization of a silicone comprising at least one radical selected from acrylate and methacrylate in a cosmetically suitable carrier medium, with the proviso that the average molar ratio of acrylate and methacrylate radicals, possibly in total, to Si atoms in the silicone is in the range from 0.014 to 0.15.

The present invention therefore provides a process for the preparation of silicone gels comprising the process steps:
A) provision of a mixture comprising
   a silicone comprising
   at least one (meth)acrylate group of the formula (I)

—O—C(O)—CR═CH₂ formula (I)
where R=—H or —CH₃,
   with the proviso that the average molar ratio of (meth)acrylate groups to Si atoms in the silicone is in a range from 0.014-0.15, and
   at least one cosmetically suitable carrier medium, in particular a cosmetic oil,
B) initiation of a radical polymerization by addition of at least one organic radical starter, preferably of an organic peroxide.

The invention further provides the silicone gels that can be prepared using the process according to the invention, as well as cosmetic formulations comprising these.

An advantage of the present invention is that the silicone gels according to the invention are notable for a velvety-silky skin feel upon application to the skin. Used as an additive in cosmetic formulations such as creams and lotions, the silicone gels moreover lead to a reduced waxy/oily skin feel. These sensory advantages are clearly noticeable even at low silicone polymer concentrations, with the effect of the silicone gels on the skin also still being detectable after a prolonged period following application. Compared to these silicone gels according to the invention, it has been found that silicone gels not in accordance with the invention which are based on silicone (meth)acrylates characterized in that the average molar ratio of (meth)acrylate groups to Si atoms is greater than 0.15 are notable for a dull, not very silky skin feel. In the case of silicone gels based on silicone (meth)acrylates having an average molar ratio of (meth)acrylate groups to Si atoms less than 0.014, it has been established that such gels not according to the invention lead to a very oily/greasy sensory impression on the skin, which is likewise undesired.

As a result of their still relatively good flowability, a further advantage of the silicone gels according to the invention is their good handleability and processability.

In a preferred embodiment of the process according to the invention, silicones are used which have at least one alkyl side chain having 2 to 30, preferably 8 to 24, particularly preferably 12 to 18, carbon atoms. This results in an increased compatibility of the resulting silicone polymers with organic carrier media, e.g. with cosmetic esters, which likewise leads to an improved skin feel of corresponding silicone gels.

The claimed process for the preparation of silicone gels comprises the process steps
A) provision of a mixture comprising
   a silicone comprising at least one (meth)acrylate group of the formula (I)

—O—C(O)—CR═CH₂ formula (I)
where R=—H or —CH₃,
   with the proviso that the average molar ratio of (meth)acrylate groups to Si atoms in the silicone is in a range from 0.014-0.015, and at least one cosmetic oil,
B) initiation of a radical polymerization by addition of at least one organic radical starter, preferably of an organic peroxide.

In connection with the present invention, the term “silicone” is to be understood as meaning compounds from the class of polyorganosiloxanes.

In connection with the present invention, the term “silicone gel” is to be understood as meaning a macroscopically homogeneous system which comprises at least one crosslinked silicone polymer on a microscopic level, the network structure of which is swollen by a second liquid phase.

In connection with the present invention, the term “cosmetic oil” is to be understood as meaning water-immiscible liquids which are suitable for the preparation of cosmetic formulations.

In connection with the present invention, water-immiscible means that, at room temperature, aqueous mixtures of cosmetic oils at oil concentrations of 0.5-99.5% by volume, based on the total mixture, lead to clouding that is already perceptible to the human eye and/or to the formation of two or more phases.

Furthermore, in connection with the present invention, cosmetic oils are preferably characterized in that they have an interfacial tension towards water of >5 mN/m. Cosmetic oils can be for example oleochemistry- or silicone chemistry-based.

In connection with the present invention, the term “organic radical starter” is to be understood as meaning organic compounds which have at least one labile, homolytically cleavable bond, such as for example an O—O— or an N═N bond and can thus disintegrate into radicals when, for example, the temperature is increased.

In connection with the present invention, the term “long-chain” is to be understood as meaning a compound which has at least 8, in particular 8 to 30, carbon atoms.

Unless otherwise stated, all percentages (%) given are percentages by mass.

In a preferred process according to the invention, the concentration of the silicone in the silicone/oil mixture is from 1 to 40% by weight, preferably from 3 to 30% by weight, particularly preferably from 5 to 25% by weight, based on the total weight of the silicone/oil mixture.

Preferred processes according to the invention are characterized in that the average molar ratio of (meth)acrylate groups to Si atoms in the silicone is in the range from 0.016 to 0.12, particularly preferably from 0.018 to 0.10, especially preferably from 0.020 to 0.075.

In the process according to the invention, preference is given to using silicones which have branched or unbranched alkyl radicals which are derived from long-chain fatty acids, such as e.g. capryl, lauryl, myrstyl, cetyl, stearyl or behenyl radicals. Very particular preference is given to using silicones substituted with cetyl radicals.

Particular preference is given in the process according to the invention to using silicones of the general formula (II):

MₐM′ₐ₁M″ₐ₂D_{b}D′_{b1}D″_{b2}T_{c}T′_{c1}T″_{c2}Q_{d} formula (II),

where

M=(R¹₃SiO_{1/2})

M′=(R²R¹₂SiO_{1/2})

M″=(R³R¹₂SiO_{1/2})

D=(R¹₂SiO_{2/2})

D′=(R²R¹SiO_{2/2})

D″=(R³R¹SiO_{2/2})

T=(R⁵SiO_{3/2})

T′=(R²SiO_{3/2})

T″=R³SiO_{3/2})

Q=(SiO_{4/2})

a=0 to 32, preferably 0 to 22, in particular >=2;

a1=0 to 32, preferably 0 to 22, in particular 0;

a2 =0 to 32, preferably 0 to 22, in particular >=2;

with the proviso that

a+a1+a2>=2, preferably=2;

b=1 to 600, preferably 10 to 500, in particular 20 to 400;

b1=0 to 80, preferably 0 to 50, in particular 1 to 20;

b2=0 to 50, preferably 0 to 20, in particular 0 to 10;

c=0 to 30, preferably 0 to 20, in particular 0;

c1=0 to 10, preferably 0 to 5, in particular 0;

c2=0 to 10, preferably 0 to 5, in particular 0;

d=0 to 15, preferably 0 to 12, in particular 0;

with the proviso that

a2+b2+c2>=1

and

R¹=independently of one another identical or different linear or branched, optionally aromatic hydrocarbon radicals having 1 to 30 carbon atoms, preferably having 1 to 6 carbon atoms, particularly preferably methyl or phenyl, in particular methyl;

R²=independently of one another identical or different linear or branched, optionally aromatic hydrocarbon radicals having 2 to 30, preferably 7 to 22, carbon atoms, preferably linear saturated hydrocarbon radicals having 6 to 30, preferably 7 to 22, carbon atoms, particularly preferably linear saturated hydrocarbon radicals having 8 to 22, in particular 12 to 18 carbon atoms, in particular dodecyl, tetradecyl, hexadecyl and octadecyl radicals;

R³=independently of one another identical or different organic radicals which have a (meth)acrylate group of the formula (I)

—O—C(O)—CR═CH₂ formula (I)

where R=—H or —CH₃;

R⁵=independently of one another identical or different radicals R¹, R² or R³, preferably R¹, in particular methyl, phenyl, dodecyl or hexadecyl.

The values a, a2, a3, b, b1, b2, c, c1, c2 and d here are statistical average values.

In a preferred embodiment of the invention, silicones according to formula (II) are used for which the following applies:

a+a1+a2=2

and

c+c1+c2+d=0.

In a further preferred embodiment of the invention, silicones according to formula (II) are used for which the following applies:

a2=2

and

c+c1+c2+d =0.

Particularly preferred silicones used in the process according to the invention have radicals which comprise at least one (meth)acrylate group of the formula (I), selected from the group:

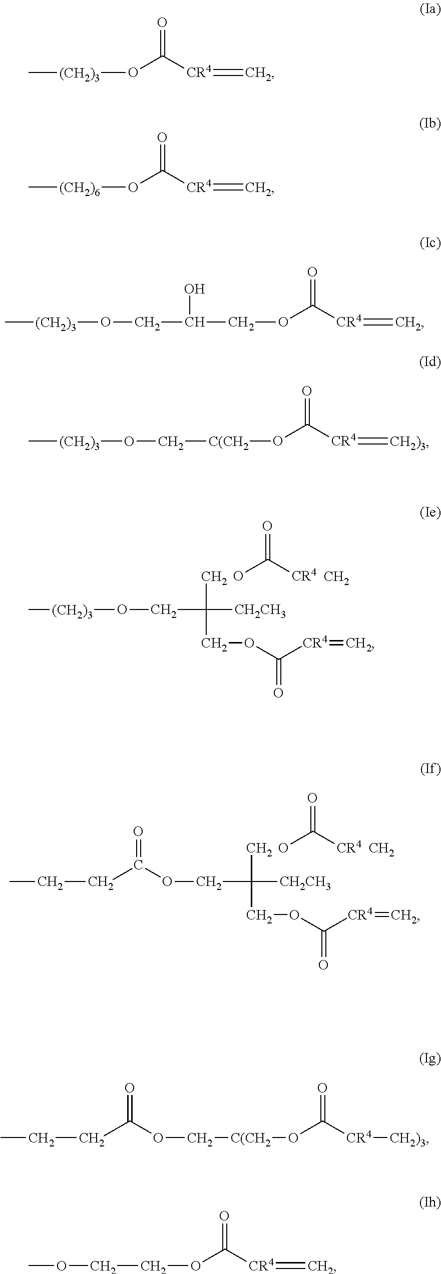

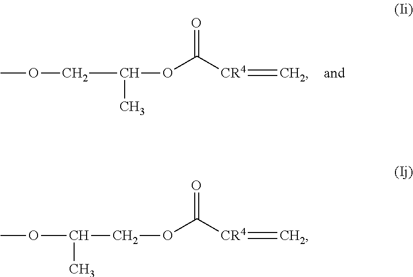

where R⁴ is hydrogen or a methyl group, preferably a methyl group. Very particularly preferably used silicones which have at least one (meth)acrylate group of the formula (I) are those which have this in the form of a radical selected from the group comprising the radicals with the formulae (Ib) and (Ie), where R⁴ is a methyl group.

In a particularly preferred embodiment of the invention, silicones according to formula (II) are used for which the following applies:

a2 =2,

b=20 to 400,

b2=1 to 20,

R¹=CH₃,

R³=—C₆H₁₂—O—C(O)—C(CH₃)═CH₂,

and a=a1=b1=c=c1=c2=d=0;

a2=2,

b=20 to 400,

R¹=CH₃,

R³=(CH₂)3—O—CH₂—C(CH₂—O—C(O)—C(CH₃)═CH2)₂—CH₂—CH₃,

and a=a1=b1=b2=c=c1=c2=d=0;

a2=2,

b=20 to 400,

R¹⁼¹ to 20,

R¹=CH₃,

R²=C6 to C24 n-alkyl,

R³=(CH₂)₃—O—CH₂—C(CH₂—O—C(O)—C(CH₃)═CH₂)₂—CH₂—CH₃,

and

a=a1=b2 =c=c1 =c2 =d=0; or

a+a2=4,

b=20 to 400,

b1=1 to 20,

c=2,

R¹=R⁵=CH₃,

R²=C6 to C24 n-alkyl,

R³=(CH₂)₃—O—CH₂—C(CH₂—O—C(O)—C(CH₃)═CH₂)₂—CH₂—CH₃,

and a1=b2=c1=c2=d=1.

In the process according to the invention, the silicones comprising at least one (meth)acrylate group of the formula (I), in particular those of the formula (II), can be used individually or as mixtures, in particular as random mixtures. Preferably, in the process according to the invention, mixtures of silicones are used, in particular those of the formula (II) in which the silicones differ as regards their structure and/or their molecular weight.

According to the invention, cosmetic oils are preferably used as cosmetically suitable carrier medium in the process according to the invention, in particular selected from the group of fatty alcohols, esters of linear fatty acids with linear or branched fatty alcohols, esters of branched fatty acids with linear or branched fatty alcohols, esters of linear fatty acids with unbranched or branched polyhydric alcohols, esters of branched fatty acids with unbranched or branched polyhydric alcohols, esters of linear fatty acids with unbranched or branched alcohols, esters of branched fatty acids with unbranched or branched alcohols, esters of alkylhydroxycarboxylic acids with linear or branched fatty alcohols. Furthermore mono-, di- or triglycerides in liquid or solid form. Furthermore esters of carboxylic acids, aromatic carboxylic acids or dicarboxylic acids with linear or branched fatty alcohols, unbranched or branched polyhydric alcohols or unbranched or branched alcohols. Furthermore, linear, cyclic or branched hydrocarbons, with or without substituents, with or without double bonds. Furthermore vegetable oils, carbonates with unbranched or branched alcohols, carbonates with unbranched or branched polyhydric alcohols, carbonates with linear or branched fatty alcohols. Furthermore ethers, with or without alkoxy groups, silicone oils, with or without organic modification. Furthermore mixtures of these oils in any desired ratios, preferably esters of linear fatty acids with linear or branched fatty alcohols, esters of branched fatty acids with linear or branched fatty alcohols, esters of linear fatty acids with unbranched or branched polyhydric alcohols, esters of branched fatty acids with unbranched or branched polyhydric alcohols, esters of linear fatty acids with unbranched or branched alcohols, esters of branched fatty acids with unbranched or branched alcohols. Furthermore mono-, di- or triglycerides in liquid or solid form. Furthermore esters of carboxylic acids, aromatic carboxylic acids or dicarboxylic acids with linear or branched fatty alcohols, unbranched or branched polyhydric alcohols or unbranched or branched alcohols. Furthermore linear, cyclic or branched hydrocarbons, with or without substituents, with or without double bonds. Furthermore vegetable oils, carbonates with unbranched or branched alcohols, carbonates with unbranched or branched polyhydric alcohols, carbonates with linear or branched fatty alcohols, particularly preferably linear, cyclic or branched hydrocarbons, with or without substituents, with or without double bonds. Furthermore carbonates with unbranched or branched alcohols, carbonates with unbranched or branched polyhydric alcohols, carbonates with linear or branched fatty alcohols. Esters of branched fatty acids with unbranched or branched polyhydric alcohols

Process step B) of the process according to the invention essentially corresponds to a conventional radical polymerization which is initiated by a peroxide. Corresponding radical polymerizations are described for example in B. Tieke, “Makromolekulare Chemie: Eine Einführung [Macromolecular Chemistry: An introduction]”, 2005, Wiley-VCH Verlag, Germany.

In a preferred process according to the invention, the organic radical starter is an organic peroxide, particularly preferably selected from the group of substances which, after the radical start, become compounds which are compatible in cosmetic products, such as e.g. hydrocarbons, alcohols or acids having 6 to 30 carbon atoms. Radical starters which can be used are in particular peroxides which are derived from organic acids, in particular fatty acids. Among these compounds which are also known as diacyl peroxides, preference is given to dilauroyl peroxide, didecanoyl peroxide and diisononanoyl peroxide. Particular preference is given to using dilauroyl peroxide as radical starter. The initiation preferably takes place by increasing the temperature.

One advantage is that such radical initiators, and decomposition products thereof are generally acceptable on health grounds, as a result of which the silicone gels can be used in cosmetic applications without concern. Particularly in the event of initiation by dilauroyl peroxide, in the course of the polymerization lauric acid is obtained as the sole decomposition product, this being a customary raw material in cosmetic applications.

The use of organic radical starters in the process according to the invention, moreover, has the advantage that they have adequate activity even without the addition of an effectiveness booster, as would be required for example when using borane initiators. This offers both process-ecological as well as process-economical advantages in the preparation of the gels.

In a further process preferred according to the invention, the organic peroxide is used in a concentration of from 0.01 to 5% by weight, preferably from 0.05 to 2% by weight, particularly preferably between 0.1 and 1% by weight, based on the total batch size.

The silicone gels which are obtainable by the process according to the invention are characterized by the fact that they have a silicone (meth)acrylate polymer network, swollen with a cosmetic oil, which originates from a silicone (meth)acrylate which is characterized in that the average molar ratio of (meth)acrylate groups to Si atoms is in the range from 0.014 to 0.15, in particular from 0.016 to 0.12, particularly preferably from 0.018 to 0.10, especially preferably from 0.020 to 0.075. The ratio of (meth)acrylate groups to Si atoms determines here the density of theoretically possible network points in the polymer network. By virtue of the process according to the invention, silicone gels within a defined crosslinking range are thus accessible which have the technical effect that they lead to a particularly velvety/silky skin feel and in so doing at the same time reduce an oily/waxy skin feel. Consequently, the silicone gels obtainable by the process according to the invention are likewise provided by the present invention.

Silicone gels preferred according to the invention have, at a shear rate of 1 l/s and a temperature of 25° C., a viscosity of less than 150 000 mPas, preferably less than 100 000 mPas, particularly preferably less than 50 000 mPas, especially preferably less than 15 000 mPas. The viscosity here can be measured by shear-rate-controlled rheology experiments at a temperature of 25° C. For this, for example, a rheometer of the type MCR 302 from Anton-Paar equipped with a plate-plate measurement geometry can be used.

Silicone gels preferred according to the invention are moreover characterized in that, in the frequency-dependent rheology test at 25° C. in the frequency range from 1-100 Hz, they have a storage modulus G′ which is always greater than the loss modulus G″. G′ and G″ here are rheological parameters known to the person skilled in the art and quantify the elastic or viscous fraction of viscoelastic liquids and are described for example in Rheokinetics: Rheological Transformations in Synthesis and Reactions of Oligomers and Polymers, A. Ya. Malkin, 1996.

This means that the silicone gels according to the invention have an excellent yield point which, for example, has an advantageous effect on their thickening properties and also their ability to stabilize dispersed constituents of cosmetic formulations. For frequency-dependent rheology tests, for example a rheometer of the type MCR 302 from Anton-Paar equipped with a plate-plate measurement geometry can be used.

Particularly preferred silicone gels are notable for the fact that, at a shear rate of 1 l/s and a temperature of 25° C., they have a viscosity of less than 10 000 mPas at the same time as in the frequency-dependent rheology test in the frequency range from 1-100 Hz having a storage modulus G′ which is always greater than the loss modulus G″. Corresponding silicone gels are characterized here by good flowability, which has an advantageous effect on their handleability and processability, but nevertheless have a pronounced yield point and therefore good thickening and stabilizing properties.

Since the silicone gels according to the invention develop excellent conditioning properties on the skin, cosmetic formulations comprising at least one silicone gel according to the invention are further provided by the present invention.

Preferred formulations according to the invention are characterized in that they comprise 1% by weight to 40% by weight, preferably 2% by weight to 30% by weight, particularly preferably 3% by weight to 24% by weight, of silicone gel according to the invention, based on the total formulation. Cosmetic care and cleansing formulations according to the invention can for example comprise at least one additional component selected from the group of

surfactants,

emollients,

emulsifiers,

coemulsifiers,

thickeners/viscosity regulators/stabilizers,

antioxidants,

hydrotropes (or polyols),

solids and fillers,

pearlescence additives,

deodorant and antiperspirant active ingredients,

insect repellents,

self-tanning agents,

preservatives,

conditioners,

perfumes,

dyes,

coloured pigments,

cosmetic active ingredients,

care additives,

superfatting agents,

solvents,

UV filters,

electrolytes,

multifunctional additives,

moisturising substances.

Substances which can be used as exemplary representatives of the individual groups are known to the person skilled in the art and can be found for example in EP2273966A1. This patent application is herewith incorporated as reference and thus forms part of the disclosure.

As regards further optional components and the amounts of these components used, reference is made expressly to the relevant handbooks known to the person skilled in the art, e.g. K. Schrader, “Grundlagen and Rezepturen der Kosmetika [Fundamentals and Formulations of Cosmetics]”, 2nd edition, page 329 to 341, Hathig Buch Verlag Heidelberg.

The amounts of the particular additives are governed by the intended use. Typical guide formulations for the respective applications are known prior art and are contained for example in the brochures of the manufacturers of the particular basic materials and active ingredients. These existing formulations can usually be adopted unchanged. If necessary, the desired modifications can, however, be undertaken without complication by means of simple experiments for the purposes of adaptation and optimization.

Since the silicone gels according to the invention can advantageously be used for the preparation of cleaning and care formulations for the home and industry, cleaning and care formulations for the home and industry comprising at least one silicone gel according to the invention are further provided by the present invention.

Preferred cleaning and care formulations according to the invention for the home and industry are textile-softening formulations and textile-care washing or cleaning compositions, dishwashing detergents, household cleaners, disinfectants, disinfectant cleaners, foam cleaners, floor cleaners, carpet cleaners, upholstery cleaners, floor care products, marble cleaners, parquet cleaners, stone and ceramic floor cleaners, wipe care compositions, stainless steel cleaners, glass cleaners, dishwashing detergents, plastic cleaners, sanitary cleaners, wood cleaners, leather cleaners, detergents, laundry care compositions, disinfectant detergents, standard detergents, gentle detergents, wool detergents, fabric softeners and impregnating compositions, with dishwashing detergents and household cleaners, in particular hand dishwashing detergents, being particularly preferred.

Preferred cleaning and care formulations according to the invention for the home and industry are characterized in that they comprise 1% by weight to 40% by weight, preferably 2% by weight to 30% by weight, particularly preferably 3% by weight to 24% by weight, of silicone gel according to the invention, based on the total formulation.

Particularly preferred cleaning and care formulations according to the invention for the home and industry additionally comprise one or more substances from the group of surfactants, builders, bleaches, bleach activators, enzymes, perfumes, perfume carriers, fluorescent agents, dyes, foam inhibitors, silicone oils, antiredeposition agents, optical brighteners, greying inhibitors, shrink preventers, anticrease agents, colour transfer inhibitors, antimicrobial active ingredients, germicides, fungicides, antioxidants, preservatives, corrosion inhibitors, antistats, bittering agents, ironing aids, phobicization and impregnation agents, swelling and slip-resist agents, neutral filling salts, and UV absorbers.

In particular, the cleaning and care formulations according to the invention for the home and industry can comprise between 0.001 and 90% by weight, particularly preferably 0.01 to 45% by weight, of one or more of the further ingredients mentioned here, where the % by weight refer to the total formulation.

Examples of surfactants that can be used are described in WO 2007/115872, page 17, line 28 to page 21, line 24.

Examples of builder substances, builders, bleaches, bleach activators, bleach catalysts and enzymes are described in WO 2007/115872, page 22, line 7 to page 25, line 26.

Antiredeposition agents, optical brighteners, greying inhibitors, colour transfer inhibitors are described by way of example in WO 2007/115872 on page 26, line 15 to page 28, line 2.

Examples of anticrease agents, antimicrobial active ingredients, germicides, fungicides, antioxidants, preservatives, antistats, ironing aids, UV absorbers are described in WO 2007/115872 on page 28, line 14 to page 30, line 22. Their explicit disclosure content in this regard forms part of this disclosure by virtue of this reference.

The silicone gels according to the invention and/or the formulations according to the invention can advantageously be used for the conditioning of skin and hair, which is thus likewise provided by the present invention.

Further advantageous uses of the silicone gels according to the invention and/or of the formulations according to the invention include cosmetic applications for example from the areas of coloured cosmetics, deodorants, wet wipes, skincare for face and body, hair care and shower products. Here, the silicone gels according to the invention can be used as binders and/or dispersion auxiliaries, for influencing the sensory profile during application of the formulation or after absorption has taken place on the skin (velvetiness/silkiness-, softness-, smoothness-, slipperiness-improving, oiliness-reducing, long-lasting skin feel), for increasing the availability of active ingredients (e.g. vitamins, fragrances, oil-soluble UV filters) from a formulation, for increasing the sun protection factor, as matting auxiliary, for moisturizing properties, as film former, and these uses are likewise provided by the present invention.

In general, the silicone gels according to the invention can be used, on account of their rheological properties, for the thickening of formulations, primarily for thickening the oil phase in W/O emulsions. The formulations resulting here are characterized by an excellent formulation stability, for example compared to identical formulations comprising cosmetic waxes, xanthan gum, silica, zinc stearate, propylene carbonate and/or stearalkonium hectorite.

The examples listed below illustrate the present invention by way of example, without any intention of restricting the invention, the scope of application of which is apparent from the entirety of the description and the claims, to the embodiments specified in the examples.

### EXAMPLES

### Materials Used in the Examples

Tegosoft® M is an isopropyl myristate which is sold by Evonik Industries as a cosmetic emollient.

Tegosoft® INI is an isononyl isononanoate which is sold by Evonik Industries as a cosmetic emollient.

Tegosoft® DEC is a diethylhexyl carbonate which is sold by Evonik Industries as a cosmetic emollient.

Tegosoft® AC is an isoamyl cocoate which is sold by Evonik Industries as a cosmetic emollient.

Tegosoft® PIB 5 Basic is a polyisobutene which is sold by Evonik Industries as a cosmetic emollient.

### Example 1 According to the Invention

### Silicone Gels Prepared from a Silicone Methacrylate with an Average Molar Ratio of Methacrylate Groups to Si Atoms of 0.055

For the preparation of these silicone gels according to the invention, a silicone according to formula (II) where a2=2, b=170, b2=8, R¹=CH₃, R³=—C₆H₁₂—O—C(O)—C(CH₃)═CH₂ and a=a1=b1=c=c1=c2=d=0 was used. 12 g of this silicone were placed in a 250 ml three-necked column equipped with CPG stirrer, reflux condenser and thermometer together with 87.5 g of a cosmetic oil and homogenized. To initiate the polymerization, in each case 0.5 g of an organic radical starter was added to the reaction mixture. Then, the reaction mixture was heated to 80° C. under a nitrogen atmosphere and with gentle stirring and held at this temperature for 2 hours. For the after-reaction, the mixture was heated for a further hour at 100° C. Then, the reaction mixture was cooled to room temperature.

TABLE 1
Reaction composition for the preparation of the silicone
gels according to the invention described in Example 1

| Sample | | |
|---|---|---|
| name | Oil phase | Radical starter |
| SG 1 | Tegosoft INI | Dibenzoyl peroxide |
| SG 2 | Tegosoft INI | tert-Butyl perbenzoate |
| SG 3 | Tegosoft INI | Dilauroyl peroxide |
| SG 4 | Tegosoft INI | Azobis(isobutyronitrile) |
| SG 5 | Tegosoft DEC | Dilauroyl peroxide |
| SG 6 | Tegosoft DEC | Dibenzoyl peroxide |

In an alternative preparation method, the silicone gels listed in Table 1 were prepared by the slow dropwise addition of the radical starter to a heated silicone macromonomer/oil mixture. For this, 12 g of the silicone dissolved in 60 g of the respective cosmetic oil were placed in a 250 ml four-necked flask equipped with CPG stirrer, reflux condenser, dropping funnel and thermometer and heated to 80° C. with gentle stirring. A solution of 0.5 g of radical starter in 27.5 g of the respective cosmetic oil was slowly added dropwise to this mixture over a period of 30 min. After the addition, the reaction mixture was held for a further 90 min at this temperature and then heated to 100° C. for a further 60 min for the after-reaction. Then, the reaction mixture was cooled to room temperature.

In a further alternative preparation process, the silicone gels described in Table 1 were prepared by the slow dropwise addition of a radical starter/silicone macromonomer mixture to a heated cosmetic oil. For this, 87.5 g of the cosmetic oil were placed in a 250 ml four-necked flask equipped with CPG stirrer, reflux condenser, dropping funnel and thermometer and heated to 80° C. with gentle stirring. 0.5 g of an organic radical starter dissolved in 12 g of the silicone was slowly added dropwise to this oil over a period of 30 min. After the addition, the reaction mixture was held at this temperature for a further 90 min and then heated to 100° C. for a further 60 min for the after-reaction. Then, the reaction mixture was cooled to room temperature.

For these silicone gels, a velvety-silky skin feel was established in the sensoric test, which could also be felt after a prolonged period following application.

### Example 2 According to the Invention

### Silicone Gels Prepared from a Silicone Methacrylate with an Average Molar Ratio of Methacrylate Groups to Si Atoms of 0.047

For the preparation of these silicone gels according to the invention, a silicone according to formula (II) where a2=2, b=83, R¹=CH₃, R³=(CH₂)₃—O—CH₂—C(CH₂—O—C(O)—C(CH₃)═CH₂)₂—CH₂—CH₃ and a=a1=b1=b2=c=c1=c2=d=0 was used. This was polymerized in selected cosmetic oils in accordance with the reaction procedures described in Example 1. Depending on the concentration of the silicone, 0.1 to 0.8 g of radical starter was added to the reaction mixture to initiate the polymerization. The precise compositions of the respective reaction mixtures are listed in Table 2. Similarly to the gels described in Example 1, the silicone gels thus prepared are characterized by a velvety-silky skin feel in the sensoric test.

TABLE 2
Reaction composition for the preparation of the silicone
gels according to the invention described in Example 2

| | Initial | | | |
|---|---|---|---|---|
| | weight of | | Initial | |
| Sample | silicone | | weight | |
| name | [g] | Oil phase | [g] | Radical starter |
| SG 7 | 5.00 | Tegosoft PIB | 95.00 | Dilauroyl peroxide |
| | | 5 Basic | | |
| SG 8 | 10.00 | Tegosoft PIB | 90.00 | Dilauroyl peroxide |
| | | 5 Basic | | |
| SG 9 | 15.00 | Tegosoft PIB | 85.00 | Dilauroyl peroxide |
| | | 5 Basic | | |
| SG 10 | 5.00 | Tegosoft DEC | 95.00 | Dilauroyl peroxide |
| SG 11 | 10.00 | Tegosoft DEC | 90.00 | Dilauroyl peroxide |
| SG 12 | 15.00 | Tegosoft DEC | 85.00 | Dilauroyl peroxide |
| SG 13 | 5.00 | Tegosoft PIB | 95.00 | Dibenzoyl peroxide |
| | | 5 Basic | | |
| SG 14 | 10.00 | Tegosoft PIB | 90.00 | Dibenzoyl peroxide |
| | | 5 Basic | | |
| SG 15 | 15.00 | Tegosoft PIB | 85.00 | Dibenzoyl peroxide |
| | | 5 Basic | | |
| SG 16 | 5.00 | Tegosoft DEC | 95.00 | Dibenzoyl peroxide |
| SG 17 | 10.00 | Tegosoft DEC | 90.00 | Dibenzoyl peroxide |
| SG 18 | 15.00 | Tegosoft DEC | 85.00 | Dibenzoyl peroxide |

### Example 3 According to the Invention

### Silicone Gels Prepared from a Hexadecyl-modified Silicone Methacrylate with an Average Molar Ratio of Methacrylate Groups to Si Atoms of 0.045

For the preparation of these silicone gels according to the invention, a silicone according to formula (II) where a2=2, b=83, b1 =4, R¹=CH₃, R³=(CH₂)₃—O—CH₂—C(CH₂—O—C(O)—C(CH₃)═CH₂)₂—CH₂—CH₃, R²=hexadecyl and a=a1=b2=c=c1=c2=d=0 was used. This was polymerized in selected cosmetic oils according to the reaction procedures described in Example 1. Depending on the concentration of the silicone, 0.1 to 0.8 g of radical starter was added to the reaction mixture to initiate the polymerization. The precise compositions of the respective reaction mixtures are shown in Table 3. The silicone gels prepared in this way were characterized by very high compatibility of the silicone polymer to the carrier medium. Moreover, a particularly velvety-silky skin feel of these silicone gels was established in the sensoric test.

TABLE 3
Reaction composition for the preparation of the silicone
gels according to the invention described in Example 3.

| | Initial | | | |
|---|---|---|---|---|
| | weight of | | Initial | |
| Sample | silicone | | weight | |
| name | [g] | Oil phase | [g] | Radical starter |
| SG 19 | 5.00 | Tegosoft PIB | 95.00 | Dilauroyl peroxide |
| | | 5 Basic | | |
| SG 20 | 7.00 | Tegosoft PIB | 93.00 | Dilauroyl peroxide |
| | | 5 Basic | | |
| SG 21 | 9.00 | Tegosoft PIB | 91.00 | Dilauroyl peroxide |
| | | 5 Basic | | |
| SG 22 | 13.00 | Tegosoft PIB | 87.00 | Dilauroyl peroxide |
| | | 5 Basic | | |
| SG 23 | 5.00 | Tegosoft DEC | 95.00 | Dilauroyl peroxide |
| SG 24 | 7.00 | Tegosoft DEC | 93.00 | Dilauroyl peroxide |
| SG 25 | 9.00 | Tegosoft DEC | 91.00 | Dilauroyl peroxide |
| SG 26 | 13.00 | Tegosoft DEC | 87.00 | Dilauroyl peroxide |
| SG 27 | 5.00 | Tegosoft M | 95.00 | Dibenzoyl peroxide |
| SG 28 | 7.00 | Tegosoft M | 93.00 | Dibenzoyl peroxide |
| SG 29 | 9.00 | Tegosoft M | 91.00 | Dibenzoyl peroxide |
| SG 30 | 13.00 | Tegosoft M | 87.00 | Dibenzoyl peroxide |
| SG 31 | 5.00 | Tegosoft AC | 95.00 | Dilauroyl peroxide |
| SG 32 | 7.00 | Tegosoft AC | 93.00 | Dilauroyl peroxide |
| SG 33 | 9.00 | Tegosoft AC | 91.00 | Dilauroyl peroxide |
| SG 34 | 13.00 | Tegosoft AC | 87.00 | Dilauroyl peroxide |

To characterize the flow behaviour of these silicone gels, rheology measurements were carried out by way of example on the samples SG 20, SG 21 and SG 22. Viscosity measurements at a shear rate of 1 l/s produced the following viscosities of 8000 mPas for sample SG 20, 25 000 mPas for sample SG 21 and 120 000 mPas for sample SG 22. Frequency-dependent rheology measurements revealed moreover that for all of these samples over the complete frequency range from 1-100 Hz, the storage modulus G′ is always significantly more than the loss modulus G″. All rheology measurements were carried out using a rheometer of the type MCR 302 from Anton-Paar equipped with a plate-plate measurement geometry at a temperature of 25° C.

### Example 4 Not According to the Invention

### Silicone Gels Prepared from a Silicone Methacrylate with an Average Molar Ratio of Methacrylate Groups to Si Atoms of 0.25

To prepare these silicone gels, a silicone according to formula (II) where a=2, b=13, b2=5, R¹32 CH₃, R³=(CH₂)₃—O—CH₂—CH(OH)—CH₂—O—C(O)—C(CH₃)═CH₂ and a1=a2=b1=c=c1=c2=d=0 was used. This was polymerized in selected cosmetic oils according to the reaction procedures described in Example 1. Depending on the concentration of the silicone, 0.1 to 0.8 g of radical starter was added to the reaction mixture to initiate the polymerization. The precise compositions of the respective reaction mixtures are summarized in Table 4. As a result of the high crosslinking density of the silicone polymer, poor compatibility of the silicone polymer with the carrier medium was observed, which firstly led to clumping of the gel and ultimately to phase separation of the sample after a storage time of several days. In contrast to the silicone gels described in Examples 1-3 according to the invention, a dull, not very velvety-silky skin feel was established in the sensoric test.

TABLE 4
Reaction composition for the preparation of the silicone gels
not according to the invention described in Example 4

| | Initial | | | |
|---|---|---|---|---|
| | weight of | | Initial | |
| Sample | silicone | | weight | |
| name | [g] | Oil phase | [g] | Radical starter |
| SG 35 | 7.00 | Tegosoft M | 93.00 | Dilauroyl peroxide |
| SG 36 | 13.00 | Tegosoft M | 87.00 | Dilauroyl peroxide |
| SG 37 | 7.00 | Tegosoft M | 93.00 | tert-Butyl perbenzoate |
| SG 38 | 13.00 | Tegosoft M | 87.00 | tert-Butyl perbenzoate |
| SG 39 | 7.00 | Tegosoft PIB | 93.00 | Dibenzoyl peroxide |
| | | 5 Basic | | |
| SG 40 | 13.00 | Tegosoft PIB | 87.00 | Dibenzoyl peroxide |
| | | 5 Basic | | |
| SG 41 | 7.00 | Tegosoft PIB | 93.00 | Dilauroyl peroxide |
| | | 5 Basic | | |
| SG 42 | 13.00 | Tegosoft PIB | 87.00 | Dilauroyl peroxide |
| | | 5 Basic | | |

### Example 5 Not According to the Invention

### Silicone Gels Prepared from a Hexadecyl-modified Silicone Methacrylate with an Average Molar Ratio of Methacrylate Groups to Si Atoms of 0.013

To prepare these silicone gels, a silicone according to formula (II) where a2=2, b=150, b1=3, R¹=CH₃, R³=—C₆H₁₂—O—C(O)—C(CH3)=CH2, R²=hexadecyl and a=a1=b2=c=c1=c2=d=0 was used. These were polymerized in selected cosmetic oils according to the reaction procedures described in Example 1. Depending on the concentration of the silicone, 0.1 to 1.5 g of radical starter was added to the reaction mixture to initiate the polymerization. The precise compositions of the respective reaction mixtures are shown in Table 5. As a consequence of the low crosslinking density of the silicone polymer, a notable influence of the silicone gels on the skin feel was established only in the case of increased concentrations of the silicone polymer. At these increased concentrations, however, it was evident that the silicone gels led primarily to a very oily/greasy sensory sensation on the skin.

TABLE 5
Reaction composition for the preparation of the silicone gels
not according to the invention described in Example 5

| | Initial | | | |
|---|---|---|---|---|
| | weight of | | Initial | |
| Sample | silicone | | weight | |
| name | [g] | Oil phase | [g] | Radical starter |
| SG 43 | 8.00 | Tegosoft AC | 92.00 | Dilauroyl peroxide |
| SG 44 | 10.00 | Tegosoft AC | 90.00 | Dilauroyl peroxide |
| SG 45 | 13.00 | Tegosoft AC | 87.00 | Dilauroyl peroxide |
| SG 46 | 20.00 | Tegosoft AC | 80.00 | Dilauroyl peroxide |
| SG 47 | 25.00 | Tegosoft AC | 75.00 | Dilauroyl peroxide |
| SG 48 | 30.00 | Tegosoft AC | 70.00 | Dilauroyl peroxide |

### Example 6 According to the Invention

### Silicone Gels Prepared from a Branched, Hexadecyl-modified Silicone Methacrylate with an Average Molar Ratio of Methacrylate Groups to Si Atoms of 0.043

For the preparation of these silicone gels according to the invention, a silicone according to formula (II) where a=2, a2=2, b=83, b1=4, c=2, R¹=R⁵=CH₃, R³=(CH₂)₃—O—CH₂—C(CH₂—O—C(O)—C(CH₃)═CH₂)₂—CH₂—CH₃, R²=hexadecyl, and al =b2 =c1=c₂₌d=0 was used. This was polymerized in selected cosmetic oils according to the reaction procedures described in Example 1. Depending on the concentration of the silicone, 0.1 to 0.8 g of radical starter was added to the reaction mixture to initiate the polymerization. The precise compositions of the respective reaction mixtures are shown in Table 6. Similarly to as in Example 3, the silicone gels prepared in this way are characterized by a very high compatibility of the silicone polymer to the carrier medium. Moreover, a particularly velvety-silky skin feel of these silicone gels was established in the sensoric test.

TABLE 6
Reaction composition for the preparation of the silicone gels
not according to the invention described in Example 4

| | Initial | | | |
|---|---|---|---|---|
| | weight of | | Initial | |
| Sample | silicone | | weight | |
| name | [g] | Oil phase | [g] | Radical starter |
| SG 49 | 7.00 | Tegosoft M | 93.00 | Dilauroyl peroxide |
| SG 50 | 13.00 | Tegosoft M | 87.00 | Dilauroyl peroxide |
| SG 51 | 7.00 | Tegosoft PIB | 93.00 | Dilauroyl peroxide |
| | | 5 Basic | | |
| SG 52 | 13.00 | Tegosoft PIB | 87.00 | Dilauroyl peroxide |
| | | 5 Basic | | |

### Comparative Example 7

### Sensory Evaluation of Cosmetic Formulations Comprising Silicone Gel according to the invention from Example 3 Compared to Silicone Gel not According to the Invention from Comparative Example 4

The formulations shown in Table 6 were prepared. The influence on the skin feel of the formulations was investigated by means of a panel test. Six people each applied a defined amount of approx. 25 μl of both formulations to a defined test field on the inside of the forearm without knowing the composition of the formulations. The formulations were spread using a finger and circular motions in the test field. After absorption had taken place, a break was taken and, after 5 minutes, the skin feel on the test field was assessed. The results of the assessment of the skin feel are shown at the end of Table 7. The properties listed are those which a majority of people described as preferred, following application of one of the two formulations compared to application of the other formulation.

TABLE 7
Formulations to Comparative Example 6, water-in-oil emulsions.
Classic W/O hot/cold lotion preparation. Phase A was dispersed
at 85° C. for 1 min at 20 500 rpm using the Ultraturrax.

| | AL 2/14 | 9a | 9b |
|---|---|---|---|
| A | ISOLAN ® GPS | 3.0 | 3.0 |
| | (Polyglyceryl-4 Diisostearate/ | | |
| | Polyhydroxystearate/Sebacate) | | |
| | Silicone gel from Ex. 3 according | 7.0 | |
| | to the invention (SG 22) | | |
| | Silicone gel from Ex. 4 not | | 7.0 |
| | according to the invention | | |
| | (SG 42) | | |
| | TEGOSOFT ® DEC (Diethylhexyl | | |
| | Carbonate) | | |
| | TEGOSOFT ® AC (Isoamyl Cocoate) | | |
| | TEGOSOFT ® PIB 5 Basic | | |
| | (Polybutene) | | |
| | Cyclopentasiloxane | 10.8 | 10.8 |
| | Hydrogenated castor oil | 0.1 | 0.1 |
| | Paracera ™ W 80 Wax (Paramelt) | 0.1 | 0.1 |
| | (Paraffin, Microcristallina cera) | | |
| B | Water | 74.8 | 74.8 |
| | Glycerin | 2.0 | 2.0 |
| | Magnesium Sulfate Heptahydrate | 1.5 | 1.5 |
| C | Euxyl ® PE 9010 (Schülke & Mayr) | 0.7 | 0.7 |
| | (Phenoxyethanol; | | |
| | Ethylhexylglycerin) | | |
| | Skin feel after 5 minutes | less waxy, | more waxy, |
| | | more | less |
| | | slippery, | slippery, |
| | | more | less |
| | | velvety/ | velvety/ |
| | | silky | silky |

The skin feel following application of the formulation comprising silicone gel from Example 3 according to the invention compared to silicone gel from Example 4 not according to the invention is less waxy and is more slippery. Overall, a more waxy and less slippery skin feel is described as dull. The velvetiness/silkiness following application of the formulation comprising silicone gel from

Example 3 according to the invention is also improved compared to silicone gel from Example 4 not according to the invention.

### Comparative Example 8

### Sensory Evaluation of Cosmetic Formulations Comprising Silicone Gel According to the Invention from Example 3 Compared to Silicone Gel not According to the Invention from Comparative Example 5

The formulations shown in Table 8 were prepared. The influence on the skin feel of the formulations was investigated by means of a panel test. Eight people each applied a defined amount of approx. 25 μl of the two formulations to a defined test field on the inside of the forearm without knowing the composition of the formulations. The formulations were spread using a finger by circular motions in the test field. After absorption had taken place, the oiliness was assessed by visual and tactile testing on the test fields.

The results of the assessment of the parameter oiliness are shown at the end of Table 8. The properties listed are those which a majority of the people described as preferred, following application of one of the two formulations compared to the application of the other formulation.

TABLE 8
Formulations to Comparative Example 7, oil-in-water
emulsions. Classic O/W hot/hot cream preparation.

| | FU 08/12 | 5a | 5b |
|---|---|---|---|
| A | TEGO ® Care 450 | 3.0 | 3.0 |
| | (Polyglyceryl-3 Methylglucose Distearate*) | | |
| | TEGIN ® M Pellets (Glyceryl Stearate) | 2.0 | 2.0 |
| | TEGO ® Alkanol 18, (Stearyl Alcohol) | 1.0 | 1.0 |
| | TEGOSOFT ® OS (Ethylhexyl Stearate) | 3.5 | 3.5 |
| | TEGOSOFT ® CT (Caprylic/Capric Triglyceride) | 3.5 | 3.5 |
| | Silicone gel from Ex. 3 according to | 12.0 | |
| | the invention (SG 34) | | |
| | Silicone gel from Ex. 5 not according | | 12.0 |
| | to the invention (SG 45) | | |
| B | Water | 74.2 | 74.2 |
| C | Methylisothiazolinone, Methylparaben, | 0.8 | 0.8 |
| | Ethylparaben (Microcare ® MEM, Thor | | |
| | Personal Care) | | |
| | Skin feel after absorption | less | more |
| | | oily | oily |

The oiliness on the skin is reduced following application of the formulation comprising silicone gel from Example 3 according to the invention compared to silicone gel from Example 5 according to the invention.

### Comparative Example 9

### Improved Stability of Cosmetic Formulations Using Silicone Gels According to the Invention as Thickener

The silicone gels according to the invention can be used as thickeners, primarily for thickening the oil phase in W/O emulsions. The formulations resulting here are notable for excellent stability, primarily as regards the freeze/thaw stability and are consequently preferred over formulation thickened in another way.

In a direct comparison, the thickening properties of the silicone gels according to the invention were compared, in a cosmetic W/O emulsion, with those of cosmetic waxes, silica, zinc stearate and a mixture consisting of dicaprylyl carbonate/stearalkonium hectorite/propylene carbonate.

TABLE 9
Formulations to Comparative Example 8. Classic W/O preparation.

| | AL 2/14 | 76 | 81 | 86* | 82 | 87* | 89* |
|---|---|---|---|---|---|---|---|
| A | ABIL ®EM90 | 2 | 2 | 2 | 2 | 2 | 2 |
| | (Cetyl PEG/PPG-10/1 Dimethicone) | | | | | | |
| | Silicone gel from Ex. 3 according to the | | 12 | | 8 | | |
| | invention (SG 20) | | | | | | |
| | TEGOSOFT ® OP | 5 | 2.8 | 4.4 | 3.6 | 5 | 5.1 |
| | (Ethylhexyl Palmitate) | | | | | | |
| | TEGOSOFT ® CT | 10 | 5.6 | 8.8 | 7.2 | 10 | 10.2 |
| | (Caprylic/Capric Triglyceride) | | | | | | |
| | TEGOSOFT ® DEC | 10 | 5.6 | 8.8 | 7.2 | 10 | 10.2 |
| | (Diethylhexyl Carbonate) | | | | | | |
| | Paracera ™ W 80 Wax (Paramelt) | 0.5 | | | | | |
| | (Paraffin, Microcristallina cera) | | | | | | |
| | Hydrogenated castor oil | 0.5 | | | | | |
| | AEROSIL ® R 972 (Silica) | | | | | 1 | |
| | Zinc Stearate | | | | | | 0.5 |
| | Cosmedia ® Gel CC (Cognis) | | | 4 | | | |
| | (Dicaprylyl Carbonate, Stearalkonium | | | | | | |
| | Hectorite, Propylene Carbonate) | | | | | | |
| B | Water | 70.7 | 70.7 | 70.7 | 70.7 | 70.7 | 70.7 |
| | Sodium Chloride | 0.5 | 0.5 | 0.5 | 0.5 | 0.5 | 0.5 |
| C | Euxyl ® PE 9010 (Schülke & Mayr) | 0.8 | 0.8 | 0.8 | 0.8 | 0.8 | 0.8 |
| | (Phenoxyethanol; Ethylhexylglycerin) | | | | | | |
| | Viscosity [Pas] | 15 | 15 | 15 | 7 | 7 | 6 |

*Phase A was dispersed for 1 min at 20 500 rpm using the Ultraturrax.

For this, formulations with a comparable viscosity were prepared. These were tested as to their freeze/thaw stability by storing the formulations overnight at −15° C. then bringing them to room temperature and repeating this process a total of three times. In the cases where the W/O emulsions were prepared with cosmetic waxes, silica, zinc stearate or a mixture consisting of dicaprylyl carbonate/stearalkonium hectorite/propylene carbonate, water separation was evident after the 3 freeze/thaw cycles. This did not occur in the cases where the W/O emulsions were thickened with the silicone gel according to the invention.

### Comparative Example 10

### Processability

As a result of their still relatively good flowability, a further advantage of the silicone gels according to the invention is their good handleability and processability. Consequently, they are distinguished from other customary silicone gels, some of which have very high viscosities, cannot be pumped and have to be scraped from the pack and homogenized and/or dispersed during processing under the influence of high shear forces.

TABLE 10
Formulations to Comparative Example 9. Classic W/O preparation.

| | AL 2/14 | 32* | 33* | 35* | 36* | 37* | 38* | 41 | 42 |
|---|---|---|---|---|---|---|---|---|---|
| A | ISOLAN ® GPS (Polyglyceryl-4 | 3.0 | 3.0 | 3.0 | 3.0 | 3.0 | 3.0 | 3.0 | 3.0 |
| | Diisostearate/Polyhydroxystearate/ | | | | | | | | |
| | Sebacate) | | | | | | | | |
| | Silicone gel from Ex. 3 according to | | | | | | | 3.0 | |
| | the invention (SG 21) | | | | | | | | |
| | Silicone gel from Ex. 3 according to | | | | | | | | 3.0 |
| | the invention (SG 25) | | | | | | | | |
| | 9041 Silicone Elastomer Blend (Dow | | 5.0 | | | | | | |
| | Corning) (Dimethicone, Dimethicone | | | | | | | | |
| | Crosspolymer) | | | | | | | | |
| | KSG-16 (Shin-Etsu) (Dimethicone, | | | 3.0 | | | | | |
| | Dimethicone/Vinyl Dimethicone | | | | | | | | |
| | Crosspolymer) | | | | | | | | |
| | 9040 Silicone Elastomer Blend (Dow | | | | 3.0 | | | | |
| | Corning) (Cyclopentasiloxane, | | | | | | | | |
| | Dimethicone Crosspolymer) | | | | | | | | |
| | KSG-15 (Shin-Etsu) | 5.0 | | | | | | | |
| | (Cyclopentasiloxane, | | | | | | | | |
| | Dimethicone/Vinyl Dimethicone | | | | | | | | |
| | Crosspolymer) | | | | | | | | |
| | Velvesil DM (Momentive) | | | | | 3.0 | | | |
| | (Dimethicone, Cetearyl Dimethicone | | | | | | | | |
| | Crosspolymer) | | | | | | | | |
| | Silsoft Silicone Gel | | | | | | 3.0 | | |
| | (Cyclopentasiloxane, Cetearyl | | | | | | | | |
| | Dimethicone/Vinyl Dimethicone | | | | | | | | |
| | Crosspolymer; Momentive) | | | | | | | | |
| | Cyclopentasiloxane | 6.4 | 6.4 | 7.4 | 7.4 | 7.4 | 7.4 | 7.4 | 7.4 |
| | TEGOSOFT ® DEC | 6.4 | 6.4 | 7.4 | 7.4 | 7.4 | 7.4 | 7.4 | 7.4 |
| | (Diethylhexyl Carbonate) | | | | | | | | |
| | Hydrogenated castor oil | 0.1 | 0.1 | 0.1 | 0.1 | 0.1 | 0.1 | 0.1 | 0.1 |
| | Paracera ™ W 80 Wax (Paramelt) | 0.1 | 0.1 | 0.1 | 0.1 | 0.1 | 0.1 | 0.1 | 0.1 |
| | (Paraffin, Microcristallina cera) | | | | | | | | |
| B | Water | 74.8 | 74.8 | 74.8 | 74.8 | 74.8 | 74.8 | 74.8 | 74.8 |
| | Glycerin | 2.0 | 2.0 | 2.0 | 2.0 | 2.0 | 2.0 | 2.0 | 2.0 |
| | Magnesium Sulfate Heptahydrate | 1.5 | 1.5 | 1.5 | 1.5 | 1.5 | 1.5 | 1.5 | 1.5 |
| C | Euxyl ® PE 9010 (Schülke & Mayr) | 0.7 | 0.7 | 0.7 | 0.7 | 0.7 | 0.7 | 0.7 | 0.7 |
| | (Phenoxyethanol; Ethylhexylglycerin) | | | | | | | | |

*Phase A was dispersed for 1 min at 20 500 rpm using the Ultraturrax.

The standard commercial silicone gels used in this example all have to be scraped from the pack using a spatula and homogenized and/or dispersed during the W/O preparation under the influence of high shear forces. In the case where the silicone gel according to the invention was used, this could be removed from the pack easily by pumping or shaking.

### Example Formulations

The majority of the formulation examples listed below are emulsions of the oil-in-water (O/W) type or water-in-oil (W/O) type. These can be produced by customary methods known to the person skilled in the art using typical stirring units. W/O emulsions are preferably prepared by slowly stirring the water phase into the oil phase with subsequent homogenization. In the case of the described O/W emulsions, oil and water phase are preferably combined without stirring and then homogenized. This can be performed in a cold/cold process or in a hot/hot process in which the homogenization takes place at approx. 70° C. The silicone gels according to the invention can be incorporated here in principle in any stage of the process. In most of the example formulations, either the incorporation into the emulsion was carried out at temperatures <60° C. or the silicone gels were initially introduced together with the oil phase. Alternatively, in some cases, a hot/cold process can also be used, in which the oil phase is heated as usual but the water phase is only heated slightly, if at all.

W/O Lotion

| | | % |
|---|---|---|
| A | ISOLAN ® GPS (Polyglyceryl-4 Diisostearate/ | 3.00 |
| | Polyhydroxystearate/Sebacate) | |
| | Silicone gel from Ex. 3 according to the invention (SG 32) | 8.00 |
| | Cyclopentasiloxane | 4.90 |
| | TEGOSOFT ® DEC (Diethylhexyl Carbonate) | 4.90 |
| | Hydrogenated castor oil | 0.10 |
| | Paracera ™ W 80 Wax (Paramelt) (Paraffin, | 0.10 |
| | Microcristallina cera) | |
| B | Water | ad 100 |
| | Glycerin | 2.00 |
| | Magnesium Sulfate Heptahydrate | 1.50 |
| C | Euxyl ® PE 9010 (Schiilke & Mayr) (Phenoxyethanol, | 0.70 |
| | Ethylhexylglycerin) | |
| D | Perfume | q.s. |

W/O Lotion

| | | % |
|---|---|---|
| A | ABIL ® EM 120 (Bis-(Glyceryl/Lauryl) Glyceryl Lauryl | 3.00 |
| | Dimethicone, Caprylic/Capric Triglyceride) | |
| | Silicone gel from Ex. 3 according to the invention (SG 28) | 12.00 |
| | Cyclopentasiloxane | 2.90 |
| | TEGOSOFT ® P (Isopropyl Palmitate) | 2.90 |
| | Hydrogenated castor oil | 0.10 |
| | Paracera ™ W 80 Wax (Paramelt) (Paraffin, | 0.10 |
| | Microcristallina cera) | |
| B | Water | ad 100 |
| | Glycerin | 2.00 |
| | Sodium Chloride | 0.80 |
| C | Euxyl ® PE 9010 (Schiilke & Mayr) (Phenoxyethanol, | 0.70 |
| | Ethylhexylglycerin) | |

W/O Lotion

| | | % | % | % | % | % | % |
|---|---|---|---|---|---|---|---|
| A | ABIL ® EM 90 (Cetyl PEG/PPG-10/1 | 3.00 | 3.00 | 3.00 | 3.00 | 3.00 | 3.00 |
| | Dimethicone) | | | | | | |
| | Silicone gel from Ex. 3 according to | 4.00 | 8.00 | 12.00 | 16.00 | 20.00 | 24.00 |
| | the invention (SG 24) | | | | | | |
| | Cyclopentasiloxane | 26.00 | 22.00 | 18.00 | 14.00 | 10.00 | 6.00 |
| B | Water | ad 100 | ad 100 | ad 100 | ad 100 | ad 100 | ad 100 |
| | Sodium Chloride | 0.50 | 0.50 | 0.50 | 0.50 | 0.50 | 0.50 |
| C | Euxyl ® PE 9010 (Schülke & Mayr) | 0.70 | 0.70 | 0.70 | 0.70 | 0.70 | 0.70 |
| | (Phenoxyethanol, Ethylhexylglycerin) | | | | | | |

W/O Lotion

| | | % | % | % | % | % | % |
|---|---|---|---|---|---|---|---|
| A | ABIL ® EM 90 (Cetyl PEG/PPG-10/1 | 2.00 | 2.00 | 2.00 | 2.00 | 2.00 | 2.00 |
| | Dimethicone) | | | | | | |
| | Silicone gel from Ex. 3 according to | 24.00 | 20.00 | 16.00 | 12.00 | 8.00 | 4.00 |
| | the invention (SG 20) | | | | | | |
| | TEGOSOFT ® OP | 0.45 | 1.20 | 2.00 | 2.80 | 3.60 | 4.40 |
| | (Ethylhexyl Palmitate) | | | | | | |
| | TEGOSOFT ® CT | 0.90 | 2.40 | 4.00 | 5.60 | 7.20 | 8.80 |
| | (Caprylic/Capric Triglyceride) | | | | | | |
| | TEGOSOFT ® DEC | 0.90 | 2.40 | 4.00 | 5.60 | 7.20 | 8.80 |
| | (Diethylhexyl Carbonate) | | | | | | |
| B | Water | ad 100 | ad 100 | ad 100 | ad 100 | ad 100 | ad 100 |
| | Sodium Chloride | 0.50 | 0.50 | 0.50 | 0.50 | 0.50 | 0.50 |
| C | Euxyl ® PE 9010 (Schülke & Mayr) | 0.80 | 0.80 | 0.80 | 0.80 | 0.80 | 0.80 |
| | (Phenoxyethanol, Ethylhexylglycerin) | | | | | | |

W/O Lotion

| | | % |
|---|---|---|
| A | ABIL ® EM 180 (Cetyl PEG/PPG-10/1 Dimethicone) | 1.50 |
| | Silicone gel from Ex. 3 according to the invention (SG 23) | 16.00 |
| | Cyclopentasiloxane | 14.00 |
| B | Water | ad 100 |
| | Sodium Chloride | 0.50 |
| C | Euxyl ® PE 9010 (Schillke & Mayr) (Phenoxyethanol, | 0.70 |
| | Ethylhexylglycerin) | |

W/O Lotion

| | | % |
|---|---|---|
| A | ABIL ® EM 180 (Cetyl PEG/PPG-10/1 Dimethicone) | 1.00 |
| | Silicone gel from Ex. 3 according to the invention (SG 29) | 8.00 |
| | TEGOSOFT ® OP (Ethylhexyl Palmitate) | 3.60 |
| | TEGOSOFT ® CT (Caprylic/Capric Triglyceride) | 7.20 |
| | TEGOSOFT ® DEC (Diethylhexyl Carbonate) | 7.20 |
| B | Water | ad 100 |
| | Sodium Chloride | 0.50 |
| C | Euxyl ® PE 9010 (Schiilke & Mayr) (Phenoxyethanol, | 0.80 |
| | Ethylhexylglycerin) | |

W/O Lotion with Urea

| | | % |
|---|---|---|
| A | ABIL ® EM 180 (Cetyl PEG/PPG-10/1 Dimethicone) | 1.50 |
| | TEGOSOFT ® G 20 (Octyldodecanol) | 2.90 |
| | TEGOSOFT ® CT (Caprylic/Capric Triglyceride) | 2.20 |
| | TEGOSOFT ® P (Isopropyl Palmitate) | 2.20 |
| | Mineral oil (30 mPas) | 3.20 |
| | Paracera ™ W 80 Wax (Paramelt) | 0.50 |
| | (Paraffin, Microcristallina cera) | |
| | Hydrogenated castor oil | 0.50 |
| | Silicone gel from Ex. 3 according to the invention (SG 25) | 12.00 |
| B | Sodium Chloride | 0.70 |
| | Glycerin | 3.00 |
| | Urea | 5.00 |
| | Triacetin (Glyceryl Triacetate) | 0.01 |
| | Water | ad 100 |
| C | Phenonip XB (Phenoxyethanol, Methylparaben, | 0.70 |
| | Propylparaben, Ethylparaben) | |

Anti-Aging Day Care Cream

| | | % |
|---|---|---|
| A | ABIL ® EM 120 (Bis-(Glyceryl/Lauryl) Glyceryl Lauryl Dimethicone, | 3.00 |
| | Caprylic/Capric Triglyceride) | |
| | TEGOSOFT ® AC (Isoamyl Cocoate) | 3.50 |
| | TEGOSOFT ® DEC (Diethylhexyl Carbonate) | 3.00 |
| | HyaCare ® Filler CL (Water; Ethylhexyl Stearate; Sodium Hyaluronate | 2.50 |
| | Crosspolymer; Polyglyceryl-4 Diisostearate/Polyhydroxystearate/Sebacate; | |
| | Sodium Isostearate) | |
| | TEGOLON ® ECO 10-10 (Nylon-10/10) | 2.00 |
| | Tocopherol | 0.50 |
| | Silicone gel from Ex. 3 according to the invention (SG 33) | 8.00 |
| B | Water | ad 100 |
| | Sodium Chloride | 0.80 |
| | Glycerin | 4.00 |
| | Butylene Glycol | 4.00 |
| | TEGO ® Pep 4-Even (Tetrapeptide-30; Glycerin) | 2.50 |
| | Sodium Ascorbyl Phosphate | 1.50 |
| | Urea | 2.50 |
| | Sodium Bisulfite (40% in water) | 0.30 |
| C | Euxyl ® PE 9010 (Schiilke & Mayr) (Phenoxyethanol, Ethylhexylglycerin) | 0.70 |

Dual-Action Wrinkle Serum

| | | % |
|---|---|---|
| A | ABIL ® EM 90 (Cetyl PEG/PPG-10/1 Dimethicone) | 1.50 |
| | ABIL ® EM 97 S (Bis-PEG/PPG-14/14 Dimethicone; Dimethicone) | 1.00 |
| | Cyclopentasiloxane | 6.00 |
| | TEGOSOFT ® DEC (Diethylhexyl Carbonate) | 5.00 |
| | HyaCare ® Filler CL (Water; Ethylhexyl Stearate; Sodium Hyaluronate | 2.50 |
| | Crosspolymer; Polyglyceryl-4 Diisostearate/Polyhydroxystearate/Sebacate; | |
| | Sodium Isostearate) | |
| | Tocopherol | 0.50 |
| | Zinc Stearate | 0.50 |
| | Silicone gel from Ex. 3 according to the invention (SG 22) | 4.00 |
| B | Water | ad 100 |
| | Glycerin | 4.00 |
| | Butylene Glycol | 4.00 |
| | Sodium Chloride | 0.80 |
| | TEGO ® Pep 4-17 (Tetrapeptide-21; Glycerin; Butylene Glycol; Water) | 0.50 |
| C | Euxyl ® PE 9010 (Schnlke & Mayr) (Phenoxyethanol, Ethylhexylglycerin) | 0.40 |

Light O/W Anti Aging Lotion (Cold Processing)

| | | % |
|---|---|---|
| A | TEGO ® Care LTP (Sorbitan Laurate; Polyglyceryl-4 Laurate; | 1.50 |
| | Dilauryl Citrate) | |
| | TEGOSOFT ® CI (Cetearyl Isononanoate) | 2.50 |
| | TEGOSOFT ® DEC (Diethylhexyl Carbonate) | 1.60 |
| | TEGOSOFT ® OP (Ethylhexyl Palmitate) | 0.50 |
| | TEGO ® Carbomer 140 (Carbomer) | 0.15 |
| | TEGO ® Carbomer 141 (Carbomer) | 0.15 |
| | Xanthan gum | 0.10 |
| | Silicone gel from Ex. 3 according to the invention (SG 27) | 10.00 |
| B | TEGO ® Cosmo C 100 (Creatine) | 0.50 |
| | Glycerin | 3.00 |
| | Water | ad 100 |
| C | Sodium Hydroxide (10% in water) | 0.90 |
| | Phenonip XB (Phenoxyethanol, Methylparaben, Propylparaben, | 0.70 |
| | Ethylparaben) | |

O/W Body Lotion

| | | % |
|---|---|---|
| A | AXOL ® C 62 Pellets (Glyceryl Stearate Citrate) | 1.50 |
| | TEGO ® Alkanol 1618 (Cetearyl Alcohol) | 1.00 |
| | TEGOSOFT ® CT (Caprylic/Capric Triglyceride) | 3.90 |
| | TEGOSOFT ® MM (Myristyl Myristate) | 1.60 |
| | Tocopheryl Acetate | 1.00 |
| | Silicone gel from Ex. 3 according to the invention (SG 19) | 8.00 |
| B | Glycerin | 5.00 |
| | GluCare ® S (Sodium Carboxymethyl Beta-Glucan) | 0.20 |
| | Water | ad 100 |
| C | TEGO ® Carbomer 141 (Carbomer) | 0.20 |
| | TEGOSOFT ® OS (Ethylhexyl Stearate) | 0.80 |
| D | Sodium Hydroxide (10% in water) | 0.60 |
| E | Alcohol | 3.00 |
| F | Microcare MEM (Dipropylene Glycol; Methylparaben; | 0.70 |
| | Ethylparaben; Water; Methylisothiazolinone) | |

Light and refreshing O/W Lotion

| | | % |
|---|---|---|
| A | Silicone gel from Ex. 3 according to the invention | 12.00 |
| | (SG 31) | |
| | ABIL ® 350 (Dimethicone) | 0.80 |
| | TEGOSOFT ® DEC (Diethylhexyl Carbonate) | 2.00 |
| | ABIL ® Care XL 80 (Bis-PEG/PPG-20/5 | 2.00 |
| | PEG/PPG-20/5 Dimethicone; Methoxy | |
| | PEG/PPG-25/4 Dimethicone; Caprylic/Capric | |
| | Triglyceride) | |
| B | Glycerin | 1.00 |
| | Water | ad 100 |
| C | TEGO ® Carbomer 341 ER (Carbomer) | 0.25 |
| | TEGOSOFT ® DEC (Diethylhexyl Carbonate) | 1.00 |
| D | Sodium Hydroxide (10% in water) | 0.70 |
| E | Euxyl ® PE 9010 (Schülke & Mayr) | 1.00 |
| | (Phenoxyethanol, Ethylhexylglycerin) | |

O/W Lotion with Light Skin Feel

| | | % |
|---|---|---|
| A | TEGO ® Care PSC 3 (Polyglyceryl-3 Dicitrate/Stearate) | 2.50 |
| | TEGOSOFT ® AC (Isoamyl Cocoate) | 3.80 |
| | TEGOSOFT ® CT (Caprylic/Capric Triglyceride) | 3.00 |
| | Prunus Amygdalus Dulcis (Sweet Almond) Oil | 2.70 |
| | Silicone gel from Ex. 3 according to the invention | 8.00 |
| | (SG 24) | |
| B | Water | ad 100 |
| | Glycerin | 3.00 |
| C | Xanthan gum | 0.50 |
| D | Sodium Hydroxide (10% in water) | 0.20 |
| E | Rokonsal BSB-N (benzyl alcohol, glycerin, benzoic acid, | 0.80 |
| | sorbic acid) | |

Wellness Body Lotion

| | | % |
|---|---|---|
| A | TEGO ® Care PSC 3 (Polyglyceryl-3 Dicitrate/Stearate) | 3.00 |
| | TEGOSOFT ® CT (Caprylic/Capric Triglyceride) | 2.70 |
| | TEGOSOFT ® OER (Oleyl Erucate) | 2.00 |
| | Prunus Amygdalus Dulcis (Sweet Almond) Oil | 3.30 |
| | Silicone gel from Ex. 3 according to the invention | 4.00 |
| | (SG 34) | |
| B | Water | ad 100 |
| | Glycerin | 3.00 |
| C | Xanthan gum | 0.50 |
| D | Sodium Hydroxide (10% in water) | 0.20 |
| E | Rokonsal BSB-N (benzyl alcohol, glycerin, benzoic acid, | 0.80 |
| | sorbic acid) | |

O/W Lotion with peptide

| | | % |
|---|---|---|
| A | TEGO ® Care PSC 3 (Polyglyceryl-3 Dicitrate/Stearate) | 3.00 |
| | TEGOSOFT ® OP (Ethylhexyl Palmitate) | 5.30 |
| | TEGOSOFT ® M (Isopropyl Myristate) | 2.60 |
| | TEGOSOFT ® CI (Cetearyl Isononanoate) | 1.10 |
| | Silicone gel from Ex. 3 according to the invention | 8.00 |
| | (SG 20) | |
| B | Water | ad 100 |
| | Glycerin | 3.00 |
| | TEGO ® Pep 4-17 (tetrapeptide-21; glycerin; | 2.50 |
| | butylene glycol; water) | |
| C | TEGO ® Carbomer 141 (Carbomer) | 0.20 |
| | TEGOSOFT ® OP (Ethylhexyl Palmitate) | 0.80 |
| D | Sodium Hydroxide (10% in water) | 0.60 |
| E | Microcare MEM (Dipropylene Glycol; Methylparaben; | 0.80 |
| | Ethylparaben; Water; Methylisothiazolinone) | |

O/W Cream

| | | % |
|---|---|---|
| A | TEGO ® Care 450 (Polyglyceryl-3 Methylglucose | 3.00 |
| | Distearate) | |
| | TEGIN ® M Pellets (Glyceryl Stearate) | 2.00 |
| | TEGO ® Alkanol 18 (Stearyl Alcohol) | 1.00 |
| | TEGOSOFT ® OS (Ethylhexyl Stearate) | 4.50 |
| | TEGOSOFT ® CT (Caprylic/Capric Triglyceride) | 4.50 |
| | Silicone gel from Ex. 3 according to the invention | 10.00 |
| | (SG 28) | |
| B | Water | ad 100 |
| | Glycerin | 3.00 |
| C | Euxyl ® PE 9010 (Schülke & Mayr) | 1.00 |
| | (Phenoxyethanol, Ethylhexylglycerin) | |

Anti-Aging O/W Cream

| | | % |
|---|---|---|
| A | TEGO ® Care 450 (Polyglyceryl-3 Methylglucose | 3.00 |
| | Distearate) | |
| | TEGIN ® M Pellets (Glyceryl Stearate) | 2.00 |
| | TEGO ® Alkanol 18 (Stearyl Alcohol) | 1.00 |
| | TEGOSOFT ® OS (Ethylhexyl Stearate) | 3.50 |
| | TEGOSOFT ® CT (Caprylic/Capric Triglyceride) | 3.50 |
| | Silicone gel from Ex. 3 according to the invention | 12.00 |
| | (SG 23) | |
| | Ceramide III (Ceramide NP) | 0.10 |
| B | Water | ad 100 |
| | Glycerin | 3.00 |
| | TEGO ® PEP 4-17 (tetrapeptide-21; glycerin; | 2.50 |
| | butylene glycol; water) | |
| | HyaCare ® 50 (Hydrolyzed Hyaluronic Acid) | 0.10 |
| C | Euxyl ® PE 9010 (Schülke & Mayr) | 1.00 |
| | (Phenoxyethanol, Ethylhexylglycerin) | |

Time Zone Anti-Aging Cream

| | | % |
|---|---|---|
| A | TEGO ® Care 450 (Polyglyceryl-3 Methylglucose | 3.00 |
| | Distearate) | |
| | TEGIN ® M Pellets (Glyceryl Stearate) | 2.00 |
| | TEGO ® Alkanol 18 (Stearyl Alcohol) | 1.00 |
| | TEGOSOFT ® CT (Caprylic/Capric Triglyceride) | 3.50 |
| | TEGOSOFT ® TN (C12-15 Alkyl Benzoate) | 3.50 |
| | Silicone gel from Ex. 3 according to the invention | 12.00 |
| | (SG 19) | |
| B | TEGO ® Pep 4-17 (tetrapeptide-21; glycerin; | 4.00 |
| | butylene glycol; water) | |
| | HyaCare ® (Sodium Hyaluronate) | 0.10 |
| | HyaCare ® 50 (Hydrolyzed Hyaluronic Acid) | 0.10 |
| | Glycerin | 3.00 |
| | Water | ad 100 |
| D | HyaCare ® Filler CL (Water; Ethylhexyl | 5.00 |
| | Stearate; Sodium Hyaluronate Crosspolymer; | |
| | Polyglyceryl-4 Diisostearate/Polyhydroxystearate/ | |
| | Sebacate; Sodium Isostearate) | |
| E | Microcare MEM (Dipropylene Glycol; Methylparaben; | 0.80 |
| | Ethylparaben; Water; Methylisothiazolinone) | |

O/W Cream

| | | % |
|---|---|---|
| A | TEGO ® Care 450 (Polyglyceryl-3 Methylglucose | 3.00 |
| | Distearate) | |
| | TEGIN ® M Pellets (Glyceryl Stearate) | 1.50 |
| | TEGO ® Alkanol 18 (Stearyl Alcohol) | 1.50 |
| | TEGOSOFT ® OS (Ethylhexyl Stearate) | 4.50 |
| | TEGOSOFT ® CT (Caprylic/Capric Triglyceride) | 3.70 |
| | Silicone gel from Ex. 3 according to the invention | 10.00 |
| | (SG 32) | |
| B | Water | ad 100 |
| | Glycerin | 3.00 |
| C | TEGO ® Carbomer 134 (Carbomer) | 0.20 |
| | TEGOSOFT ® CT (Caprylic/Capric Triglyceride) | 0.80 |
| D | Euxyl ® PE 9010 (Schülke & Mayr) | 1.00 |
| | (Phenoxyethanol, Ethylhexylglycerin) | |

O/W Cream

| | | % |
|---|---|---|
| A | TEGO ® Care 165 (Glyceryl Stearate; PEG-100 | 6.00 |
| | Stearate) | |
| | TEGIN ® M Pellets (Glyceryl Stearate) | 2.00 |
| | TEGO ® Alkanol 18 (Stearyl Alcohol) | 1.00 |
| | TEGOSOFT ® OS (Ethylhexyl Stearate) | 4.50 |
| | TEGOSOFT ® CT (Caprylic/Capric Triglyceride) | 4.50 |
| | Silicone gel from Ex. 3 according to the invention | 10.00 |
| | (SG 27) | |
| B | Water | ad 100 |
| | Glycerin | 3.00 |
| C | Microcare MEM (Dipropylene Glycol; Methylparaben; | 0.80 |
| | Ethylparaben; Water; Methylisothiazolinone) | |

Anti-Aging O/W Cream

| | | % |
|---|---|---|
| A | TEGO ® Care 165 (Glyceryl Stearate; PEG-100 | 6.00 |
| | Stearate) | |
| | TEGIN ® M Pellets (Glyceryl Stearate) | 2.00 |
| | TEGO ® Alkanol 18 (Stearyl Alcohol) | 1.00 |
| | TEGOSOFT ® OS (Ethylhexyl Stearate) | 5.50 |
| | TEGOSOFT ® CT (Caprylic/Capric Triglyceride) | 5.50 |
| | Silicone gel from Ex. 3 according to the invention | 8.00 |
| | (SG 21) | |
| | Ceramide III (Ceramide NP) | 0.10 |
| B | Water | ad 100 |
| | Glycerin | 3.00 |
| | TEGO ® PEP 4-17 (tetrapeptide-21; glycerin; | 2.50 |
| | butylene glycol; water) | |
| | HyaCare ® 50 (Hydrolyzed Hyaluronic Acid) | 0.10 |
| C | Microcare MEM (Dipropylene Glycol; Methylparaben; | 0.80 |
| | Ethylparaben; Water; Methylisothiazolinone) | |

O/W Urea Softcream

| | | % |
|---|---|---|
| A | TEGO ® Care PSC 3 (Polyglyceryl-3 Dicitrate/Stearate) | 3.00 |
| | TEGIN ® M Pellets (Glyceryl Stearate) | 1.50 |
| | TEGO ® Alksnol 1618 (Cetearyl Alcohol) | 1.00 |
| | TEGOSOFT ® CT (Caprylic/Capric Triglyceride) | 3.90 |
| | TEGOSOFT ® OS (Ethylhexyl Stearate) | 2.00 |
| | TEGOSOFT ® DC (Decyl Cocoate) | 1.60 |
| | Silicone gel from Ex. 3 according to the invention | 4.00 |
| | (SG 26) | |
| B | Water | ad 100 |
| | Glycerin | 3.00 |
| C | Water | 10.00 |
| | Urea | 10.00 |
| | Triacetin (Glyceryl Triacetate) | 0.01 |
| D | Microcare MEM (Dipropylene Glycol; Methylparaben; | 0.80 |
| | Ethylparaben; Water; Methylisothiazolinone) | |

O/W Eye Cream with low SPF (UVA)

| | | % |
|---|---|---|
| A | TEGO ® Care PSC 3 (Polyglyceryl-3 Dicitrate/Stearate) | 3.00 |
| | TEGIN ® M Pellets (Glyceryl Stearate) | 2.50 |
| | TEGO ® Alkanol 18 (Stearyl Alcohol) | 1.50 |
| | TEGOSOFT ® CT (Caprylic/Capric Triglyceride) | 0.30 |
| | TEGOSOFT ® XC (Phenoxyethyl Caprylate) | 4.00 |
| | TEGOSOFT ® TIS (Triisostearin) | 1.00 |
| | TEGOSOFT ® CR (Cetyl Ricinoleate) | 0.50 |
| | HyaCare ® Filler CL (Water; Ethylhexyl Stearate; | 3.00 |
| | Sodium Hyaluronate Crosspolymer; Polyglyceryl-4 | |
| | Diisostearate/Polyhydroxystearate/Sebacate; | |
| | Sodium Isostearate) | |
| | Persea Gratissima (Avocado) Oil | 2.00 |
| | Octocylene | 4.00 |
| | Ethylhexyl Methoxycinnamate | 2.00 |
| | Diethylamino Hydroxybenzoyl Hexyl Benzoate | 1.70 |
| | Tocopheryl Acetate | 0.50 |
| | Silicone gel from Ex. 3 according to the invention | 4.00 |
| | (SG 30) | |
| B | Water | ad 100 |
| | Glycerin | 3.00 |
| C | Microcare MEM (Dipropylene Glycol; Methylparaben; | 0.80 |
| | Ethylparaben; Water; Methylisothiazolinone) | |

Si/W Emulsion

| | | % |
|---|---|---|
| A | Silicone gel from Ex. 3 according to the invention | 10.00 |
| | (SG 20) | |
| | ABIL ® 350 (Dimethicone) | 0.80 |
| | Dimethicone (Wacker Belsil DM 5) | 2.00 |
| | ABIL ® Care XL 80 (Bis-PEG/PPG-20/5 | 2.00 |
| | PEG/PPG-20/5 Dimethicone; Methoxy | |
| | PEG/PPG-25/4 Dimethicone; Caprylic/Capric | |
| | Triglyceride) | |
| B | Glycerin | 1.00 |
| | Water | ad 100 |
| C | TEGO ® Carbomer 341 ER (Carbomer) | 0.25 |
| | Dimethicone (Wacker Belsil DM 5) | 1.00 |
| D | Sodium Hydroxide (10% in water) | 0.70 |
| E | Euxyl ® PE 9010 (Schülke & Mayr) | 1.00 |
| | (Phenoxyethanol, Ethylhexylglycerin) | |

Men's Care Gel

| | | % |
|---|---|---|
| A | TEGOSOFT ® OER (Oleyl Erucate) | 1.00 |
| | TEGOSOFT ® TIS (Triisostearin) | 1.00 |
| | Silicone gel from Ex. 3 according to the invention | 4.00 |
| | (SG 22) | |
| B | Water | ad 100 |
| | Glycerin | 5.00 |
| | TEGO ® Carbomer 341 ER (Carbomer) | 0.50 |
| | HyaCare ® 50 (Hydrolyzed Hyaluronic Acid) | 0.10 |
| C | Sodium Hydroxide (10% in water) | 1.50 |
| D | Microcare MEM (Dipropylene Glycol; Methylparaben; | 0.80 |
| | Ethylparaben; Water; Methylisothiazolinone) | |

Deodorant Cream for sensitive skin

| | | % |
|---|---|---|
| A | TEGO ® Care PBS 6 (Polyglyceryl-6 Stearate, | 3.00 |
| | Polyglyceryl-6 Behenate) | |
| | TEGIN ® M Pellets (Glyceryl Stearate) | 1.00 |
| | TEGO ® Alkanol 18 (Stearyl Alcohol) | 2.00 |
| | TEGOSOFT ® E (PPG-15 Stearyl Ether) | 11.50 |
| | Persea Gratissima (Avocado) Oil | 2.00 |
| | TEGODEO ® PY 88 G (Zinc Ricinoleate) | 1.00 |
| | TEGO ® Cosmo P 813 (Polyglyceryl-3 Caprylate) | 0.50 |
| | Triethyl Citrate | 1.00 |
| | Silicone gel from Ex. 3 according to the invention | 4.00 |
| | (SG 33) | |
| B | Natrosol 250 HHR (Hydroxyethyl Cellulose) | 1.00 |
| | Water | ad 100 |
| C | Microcare MEM (Dipropylene Glycol; Methylparaben; | 0.80 |
| | Ethylparaben; Water; Methylisothiazolinone) | |

HyaCare ® Lip Filler

| | | % |
|---|---|---|
| A | TEGOSOFT ® G 20 (Octyldodecanol) | 12.40 |
| | TEGOSOFT ® CT (Caprylic/Capric Triglyceride) | 8.80 |
| | TEGOSOFT ® OER (Oleyl Erucate) | 2.80 |
| | TEGOSOFT ® MM (Myristyl Myristate) | 5.00 |
| | Ricinus Communis (Castor) Seed Oil | 32.20 |
| | CREMERLIN ® pura (Olus (Vegetable) Oil) | 10.00 |
| | BW Ester BW 67 (Stearyl Beeswax; Behenyl Beeswax) | 6.50 |
| | Kahlwax 1899 (Ozokerite) | 6.00 |
| | Kahlwax 8104 (Cera Alba) | 5.00 |
| | Kahlwax 2039L (Euphorbia Cerifera (Candellila) Wax) | 3.00 |
| | Silicone gel from Ex. 3 according to the invention (SG 25) | 8.00 |
| B | Tocopherol | 0.10 |
| | Perfume Vanillea | 0.20 |

W/O Foundation

| | | % |
|---|---|---|
| A | ABIL ® EM 120 (Bis-(Glyceryl/Lauryl) Glyceryl | 4.00 |
| | Lauryl Dimethicone, Caprylic/Capric Triglyceride) | |
| | TEGOSOFT ® XC (Phenoxyethyl Caprylate) | 3.50 |
| | Cyclopentasiloxane | 3.50 |
| | Isododecane | 3.50 |
| | Sicovit Brown 70 E 172 (CI 77491; CI 77492; CI | 2.50 |
| | 77499 (Iron Oxides)) | |
| | Hombitan AC 360 (CI 77891; Titanium Dioxide; | 4.00 |
| | Alumina; Triethyoxycaprylylsilane) | |
| | Talc | 2.00 |
| | Silicone gel from Ex. 3 according to the | 4.00 |
| | invention (SG 26) | |
| B | Cosmedia Gel CC (Dicaprylyl Carbonate; | 4.00 |
| | Stearalkonium Hectorite; Propylene Carbonate) | |
| C | Water | ad 100 |
| | Glycerin | 5.00 |
| | Sodium Chloride | 2.00 |
| D | Boroneige 1201 (Boron Nitride) | 5.00 |
| | Phenonip XB (Phenoxyethanol, Methylparaben, | 0.70 |
| | Propylparaben, Ethylparaben) | |

Mattifying Compact Cream Make-up

| | | % |
|---|---|---|
| A | TEGOSOFT ® AC (Isoamyl Cocoate) | 23.70 |
| | TEGOSOFT ® CR (Cetyl Ricinoleate) | 5.00 |
| | TEGOSOFT ® OP (Ethylhexyl Palmitate) | 8.00 |
| | ABIL ® Wax 9840 (Cetyl Dimethicone) | 1.00 |
| | TEGOSOFT ® XC (Phenoxyethyl Caprylate) | 13.00 |
| | Microwax 1899 (Ozokerite) | 2.00 |
| | Kahlwax 6607 (Helianthus Annuus (Sunflower Seed) Wax) | 4.00 |
| | Silicone gel from Ex. 3 according to the invention (SG 31) | 8.00 |
| B | TiO₂ Kronos 1171 (Titanium Dioxide) | 12.00 |
| | Sicovit Yellow 10E (Yellow Iron Oxide) | 1.00 |
| | Sicovit Red 30E (Red Iron Oxide) | 0.30 |
| | Sicovit Brown 70E (Brown Iron Oxide) | 0.60 |
| | Sicovit Black 80E (Black Iron Oxide) | 0.20 |
| | TEGOLON ® ECO 10-10 (Nylon-10/10) | 20.00 |
| C | Dermosoft GMCY (Glyceryl Caprylate) | 1.00 |
| | Perfume Shea butter & Argan oil | 0.20 |

Color Control Fluid SPF 25

| | | % |
|---|---|---|
| A | ABIL ® EM 120 (Bis-(Glyceryl/Lauryl) Glyceryl | 4.00 |
| | Lauryl Dimethicone, Caprylic/Capric Triglyceride) | |
| | TEGOSOFT ® DEC (Diethylhexyl Carbonate) | 6.20 |
| | Cyclopentasiloxane | 6.00 |
| | Uvinul A Plus B (Ethylhexyl Methoxycinnamate; | 10.00 |
| | Diethylamino Hydroxybenzoyl Hexyl Benzoate) | |
| | TEGOLON ® 12-20 (Nylon-12) | 2.00 |
| | Micro Talc IT Extra-AW (Talc) | 2.00 |
| | Hombitan AC 360 (CI 77891; Titanium Dioxide; | 5.00 |
| | Alumina; Triethyoxycaprylylsilane) | |
| | Unipure Yellow LC 182 | 0.50 |
| | Unipure Red LC 381 | 0.20 |
| | Unipure Black LC 989 | 0.10 |
| | Timiron Splendid Gold (Titanium Dioxide; Mica; | 0.25 |
| | Silica) | |
| | Silicone gel from Ex. 3 according to the | 4.00 |
| | invention (SG 29) | |
| B | Cosmedia Gel CC (Dicaprylyl Carbonate; Stearalkonium | 3.00 |
| | Hectorite; Propylene Carbonate) | |
| C | Water | ad 100 |
| | Glycerin | 3.00 |
| | Sodium Chloride | 1.50 |
| | TEGO ® Cosmo C 250 (1-Methylhydantoin-2-Imide) | 1.00 |
| | TEGO ® Pep 4-even (Tetrapeptide-30; Glycerin) | 2.00 |
| | TEGO ® Stemlastin (Algae Extract) | 1.00 |
| D | Euxyl ® K 900 (Schülke & Mayr GmbH) (Benzyl | 1.00 |
| | Alcohol, Ethylhexylglycerin, Tocopherol) | |

Skin Beautifier BB Cream SPF 15

| | | % |
|---|---|---|
| A | TEGO ® Care 450 (Polyglyceryl-3 | 3.00 |
| | Methylglucose Distearate) | |
| | TEGIN ® M Pellets (Glyceryl Stearate) | 2.50 |
| | TEGO ® Alkanol 18 (Stearyl Alcohol) | 1.50 |
| | TEGOSOFT ® DEC (Diethylhexyl Carbonate) | 2.90 |
| | HyaCare ® Filler CL (Water; Ethylhexyl | 2.00 |
| | Stearate; Sodium Hyaluronate Crosspolymer; | |
| | Polyglyceryl-4 Diisostearate/ | |
| | Polyhydroxystearate/Sebacate; Sodium | |
| | Isostearate) | |
| | Phytosphingosine | 0.10 |
| | Ethylhexyl Methoxycinnamate | 5.00 |
| | Diethylamino Hydroxybenzoyl Hexyl Benzoate | 3.00 |
| | Silicone gel from Ex. 3 according to the | 4.00 |
| | invention (SG 34) | |
| B | Hombitan AC 360 (CI 77891; Titanium Dioxide; | 3.00 |
| | Alumina; Triethyoxycaprylylsilane) | |
| | Micro Talc IT Extra-AW (Talc) | 2.00 |
| | Unipure Yellow LC 182 | 0.36 |
| | Unipure Red LC 381 | 0.12 |
| | Unipure Black LC 989 | 0.08 |
| | TEGOSOFT ® AC (Isoamyl Cocoate) | 4.44 |
| | TEGOSOFT ® XC (Phenoxyethyl Caprylate) | 4.00 |
| | TEGOLON ® ECO 10-10 (Nylon-10/10) | 3.00 |
| C | Water | ad 100 |
| | Glycerin | 3.00 |
| | HyaCare ® 50 (Hydrolyzed Hyaluronic Acid) | 0.10 |
| | TEGO ® Pep 4-17 (tetrapeptide-21; glycerin; | 2.00 |
| | butylene glycol; water) | |
| D | Alcohol | 3.00 |
| E | Microcare MEM (Dipropylene Glycol; | 0.80 |
| | Methylparaben; Ethylparaben; Water; | |
| | Methylisothiazolinone) | |

Face Powder

| | | % |
|---|---|---|
| A | Talc | 76.00 |
| | Magnesium Stearate | 2.50 |
| | Ronaflair M Spheres (Mica; Silica) | 10.00 |
| | Sicovit Brown 70E (Brown Iron Oxide) | 3.00 |
| | TEGOLON ® ECO 10-10 (Nylon-10/10) | 5.00 |
| B | Silicone gel from Ex. 3 according to the | 3.00 |
| | invention (SG 22) | |
| | Dermosoft LP (Caprylyl Glycol; Glyceryl | 0.50 |
| | Caprylate; Glycerin; Phenyl Propanol; Water) | |

Eye Shadow blue

| | | % |
|---|---|---|
| A | Talc | 43.50 |
| | Magnesium Stearate | 5.00 |
| | Sicovit Black 80E 172 (CI 77499; Black Iron Oxide) | 1.00 |
| | Sicomet Blue P 77007 (CI 77007; Ultramarines) | 14.00 |
| | TEGOLON ® ECO 10-10 (Nylon-10/10) | 5.00 |
| B | Colorona Light Blue (Mica; Titanium Dioxide; | 25.00 |
| | Ferric Ferrocyanide) | |
| C | ABIL ® 350 (Dimethicone) | 3.00 |
| | Dermosoft LP (Caprylyl Glycol; Glyceryl Caprylate; | 0.50 |
| | Glycerin; Phenyl Propanol; Water) | |
| | Silicone gel from Ex. 3 according to the | 3.00 |
| | invention (SG 30) | |

W/O Sun Lotion with medium SPF

| | | % |
|---|---|---|
| A | ABIL ® EM 90 (Cetyl PEG/PPG-10/1 Dimethicone) | 2.50 |
| | Paracera ™ W 80 Wax (Paramelt) (Paraffin, | 0.20 |
| | Microcristallina cera) | |
| | Hydrogenated Castor Oil | 0.20 |
| | Silicone gel from Ex. 3 according to the | 10.00 |
| | invention (SG 21) | |
| | TEGOSOFT ® DEC (Diethylhexyl Carbonate) | 3.80 |
| | TEGOSOFT ® CT (Caprylic/Capric Triglyceride) | 4.90 |
| | TEGOSOFT ® XC (Phenoxyethyl Caprylate) | 4.90 |
| | Octocrylene | 4.00 |
| | Butyl Methoxydibenzoylmethane | 2.50 |
| | Ethylhexyl Methoxycinnamate | 3.00 |
| | Bis-Ethylhexyloxyphenol Methoxyphenyl Triazine | 2.00 |
| B | Water | ad 100 |
| | Glycerin | 3.00 |
| | Sodium Chloride | 0.50 |
| | Phenylbenzimidazolesulphonic acid (20% in water) | 5.00 |
| C | Euxyl ® PE 9010 (Schülke & Mayr) | 0.70 |
| | (Phenoxyethanol, Ethylhexylglycerin) | |

Low viscous W/O Sun Care Lotion SPF 30

| | | % |
|---|---|---|
| A | Diethylamino Hydroxybenzoyl Hexyl Benzoate | 9.00 |
| | Ethylhexyl Methoxycinnamate | 8.00 |
| | Octocrylene | 4.00 |
| B | ISOLAN ® GPS (Polyglyceryl-4 Diisostearate/ | 2.00 |
| | Polyhydroxystearate/Sebacate) | |
| | TEGOLON ® ECO 10-10 (Nylon-10/10) | 2.00 |
| | Sepigel 305 (Polyacrylamide, C13-14 Isoparaffin, | 1.25 |
| | Laureth-7) | |
| | EUXYL ® K 300 (Schülke & Mayr) | 0.50 |
| | (Phenoxyethanol; Methylparaben; Ethylparaben; | |
| | Butylparaben; Propylparaben; Isobutylparaben) | |
| | Aerosil R 812 S (Silica Silyate) | 0.50 |
| | Silicone gel from Ex. 3 according to the | 4.00 |
| | invention (SG 34) | |
| C | Water | ad 100 |
| | Glycerin | 5.00 |
| | VARISOFT ® 300 (Cetrimonium Chloride) | 1.00 |
| | Alcohol | 0.30 |
| D | Perfume | q.s. |

O/W Sun Protection Lotion with high SPF

| | | % |
|---|---|---|
| A | AXOL ® C 62 Pellets (Glyceryl Stearate | 2.50 |
| | Citrate) | |
| | TEGIN ® M Pellets (Glyceryl Stearate) | 1.00 |
| | TEGO ® Alkanol 1618 (Cetearyl Alcohol) | 0.50 |
| | TEGOSOFT ® XC (Phenoxyethyl Caprylate) | 6.00 |
| | Diisopropyl Adipate | 2.00 |
| | Diethylamino Hydroxybenzoyl Hexyl Benzoate | 4.00 |
| | Octocrylene | 8.00 |
| | Butyl Methoxydibenzoylmethane | 1.50 |
| | Ethylhexyl Salicylate | 5.00 |
| | Ethylhexyl Triazone | 2.00 |
| | Ethylhexyl Methoxycinnamate | 2.00 |
| | REWOPAL ® PIB 1000 (Polyisobutene) | 2.00 |
| | Silicone gel from Ex. 3 according to the | 4.00 |
| | invention (SG 26) | |
| B | Water | ad 100 |
| | Glycerin | 3.00 |
| C | TEGOSOFT ® XC (Phenoxyethyl Caprylate) | 0.80 |
| | TEGO ® Carbomer 141 (Carbomer) | 0.20 |
| D | Sodium Hydroxide (10% in water) | 0.60 |
| E | TEGOLON ® ECO 10-10 (Nylon-10/10) | 5.00 |
| F | Microcare MEM (Dipropylene Glycol; | 0.80 |
| | Methylparaben; Ethylparaben; Water; | |
| | Methylisothiazolinone) | |

O/W Sun Protection Lotion Cold Processing

| | | % |
|---|---|---|
| A | TEGO ® Care LTP (Sorbitan Laurate; | 2.00 |
| | Polyglyceryl-4 Laurate; Dilauryl Citrate) | |
| | TEGOSOFT ® DC (Decyl Cocoate) | 2.00 |
| | TEGOSOFT ® TN (C12-15 Alkyl Benzoate) | 1.50 |
| | TEGOSOFT ® OP (Ethylhexyl Palmitate) | 0.60 |
| | Ethylhexyl Methoxycinnamate | 5.00 |
| | Butyl Methoxydibenzoylmethane | 2.00 |
| | Octocrylene | 3.00 |
| | Persea Gratissima (Avocado) Oil | 1.00 |
| | TEGO ® Carbomer 141 (Carbomer) | 0.15 |
| | TEGO ® Carbomer 140 (Carbomer) | 0.15 |
| | Xanthan gum | 0.10 |
| | Silicone gel from Ex. 3 according to the | 4.00 |
| | invention (SG 33) | |
| B | Glycerin | 3.00 |
| | Water | ad 100 |
| C | Sodium Hydroxide (10% in water) | 0.90 |
| D | Phenonip XB (Phenoxyethanol, Methylparaben, | 1.00 |
| | Propylparaben, Ethylparaben) | |

O/W After Sun Lotion

| | | % |
|---|---|---|
| A | TEGO ® Care PS (Methyl Glucose Sesquistearate) | 2.00 |
| | TEGOSOFT ® OS (Ethylhexyl Stearate) | 2.50 |
| | Mineral oil (30 mPas) | 2.50 |
| | Tocopheryl Acetate | 0.50 |
| | Silicone gel from Ex. 3 according to the | 8.00 |
| | invention (SG 21) | |
| B | Glycerin | 3.00 |
| | Water | ad 100 |
| | Panthenol | 0.50 |
| C | Mineral oil (30 mPas) | 0.80 |
| | TEGO ® Carbomer 141 (Carbomer) | 0.20 |
| D | Sodium Hydroxide (10% in water) | 0.40 |
| E | Euxyl ® PE 9010 (Schülke & Mayr) | 1.00 |
| | (Phenoxyethanol, Ethylhexylglycerin) | |

Moisturizing After Sun Lotion

| | | % |
|---|---|---|
| A | TEGO ® Care PBS 6 (Polyglyceryl-6 Stearate, | 3.00 |
| | Polyglyceryl-6 Behenate) | |
| | TEGIN ® M Pellets (Glyceryl Stearate) | 0.35 |
| | TEGO ® Alkanol 1618 (Cetearyl Alcohol) | 0.65 |
| | Butyrospermum parkii (Shea) Butter | 3.00 |
| | TEGOSOFT ® CT (Caprylic/Capric Triglyceride) | 3.50 |
| | TEGOSOFT ® P (Isopropyl Palmitate) | 3.00 |
| | Silicone gel from Ex. 3 according to the | 4.00 |
| | invention (SG 30) | |
| B | Water | ad 100 |
| | Glycerin | 5.00 |
| | Triacetin (Glyceryl Triacetate) | 0.20 |
| C | Xanthan gum | 0.50 |
| D | Urea (50% in water) | 40.00 |
| E | Euxyl ® PE 9010 (Schülke & Mayr) | 0.70 |
| | (Phenoxyethanol, Ethylhexylglycerin) | |

Suspension Antiperspirant Roll-on

| | | % |
|---|---|---|
| A | Cyclopentasiloxane | 57.00 |
| | Silicone gel from Ex. 3 according to the invention (SG 20) | 10.00 |
| | Quaternium-18 Hectorite | 11.50 |
| | AEROSIL ® R 972 (Silica) | 0.50 |
| | Aluminium Chlorohydrate | 20.00 |
| B | Perfume | 1.00 |

Suspension Antiperspirant Roll-on

| | | % |
|---|---|---|
| A | Cyclopentasiloxane, Disteardimonium Hectorite, | 3.00 |
| | Propylene Carbonate (Bentone Gel ® VS-5 PC V) | |
| | Cyclopentasiloxane | 60.00 |
| | Silicone gel from Ex. 3 according to the | 10.00 |
| | invention (SG 24) | |
| | ABIL ® 100 (Dimethicone) | 4.00 |
| | Ethanol | 2.00 |
| B | Aluminium Zirconium Tetrachlorohydrex GLY | 20.00 |
| | (Reach AZP-908 O) | |
| C | Perfume | 1.00 |

All references cited herein are fully incorporated by reference in their entirety. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A process for the preparation of silicone gels, comprising the steps:
a) providing a mixture comprising:
i) a silicone comprising:
at least one (meth)acrylate group of formula (I)
—O—C(O)—CR═CH₂ formula (I)
where R=—H or —CH₃,
with the proviso that the average molar ratio of (meth)acrylate groups to Si atoms in the silicone is in a range from 0.014-0.15; and
ii) at least one cosmetically suitable carrier medium;
b) initiating a free-radical polymerization by adding at least one organic radical starter.

2. The process of claim 1, wherein the silicone used has at least one alkyl side chain having 2 to 30 carbon atoms.

3. The process of claim 1, wherein the concentration of the silicone in the silicone/oil mixture is from 1 to 40% by weight, based on the total weight of the silicone/oil mixture.

4. The process of claim 1, wherein the average molar ratio of (meth)acrylate groups to Si atoms in the silicone is in the range of from 0.020 to 0.075.

5. The process of claim 1, wherein:
a) the silicone used has at least one alkyl side chain having 12 to 18, carbon atoms;
b) the concentration of the silicone in the silicone/oil mixture is 5 to 25% by weight, based on the total weight of the silicone/oil mixture;
c) the average molar ratio of (meth)acrylate groups to Si atoms in the silicone is in the range of from 0.020 to 0.075.

6. The process of claim 1, wherein the silicone has branched or unbranched alkyl radicals which are derived from a long-chain fatty acid.

7. The process of claim 1, wherein the silicone has cetyl radicals.

8. The process of claim 1, wherein the silicone corresponds to general formula (II):
MₐM′ₐ₁M″ₐ₂D_{b}D′_{b1}D″_{b2}T_{c}T′_{c1}T″_{c2}Q_{d} formula (II),
wherein
M=(R¹₃SiO_{1/2})
M′=(R²R¹₂SiO_{1/2})
M″=(R³R¹₂SiO_{1/2})
D=(R¹₂Si O_{2/2})
D′=(R²R¹SiO_{2/2})
D″=(R³R¹SiO_{2/2})
T=(R⁵SiO_{3/2})
T′=(R²SiO_{3/2})
T″=R³SiO_{3/2})
Q=(Si O_{4/2})
a=0 to 32,
a1=0 to 32,
a2=0 to 32,
with the proviso that
a+a1+a2>=2,
b=1 to 600,
b1=0 to 80,
b2=0 to 50,
c=0 to 30,
c1=0 to 10,
c2=0 to 10,
d=0 to 15,
with the proviso that
a2+b2+c2>=1
and
R¹=independently of one another, identical or different linear or branched, optionally aromatic, hydrocarbon radicals having 1 to 30 carbon atoms;
R² =independently of one another, identical or different linear or branched, optionally aromatic, hydrocarbon radicals having 2 to 30;
R³=independently of one another, identical or different organic radicals which have a (meth)acrylate group of the formula (I):
—O—C(O)—CR═CH₂ formula (I)
where R=—H or —CH₃;
R⁵=independently of one another, identical or different radicals R¹, R² or R³.

9. The process of claim 1, wherein the silicone corresponds to general formula (II):
MₐM′ₐ₁M″ₐ₂D_{b}D′_{b1}D″_{b2}T_{c}T′_{c1}T″_{c2}Q_{d} formula (II),
wherein
M=(R¹₃SiO_{1/2})
M′=(R²R¹₂SiO_{1/2})
M″=(R³R¹₂SiO_{1/2})
D=(R¹₂SiO_{2/2})
D′=(R²R¹SiO_{2/2})
D″=(R³R¹ SiO_{2/2})
T=(R⁵SiO_{3/2})
T′=(R²SiO_{3/2})
T″=R³SiO_{3/2})
Q=(SiO_{4/2})
a=0 to 32,
a1=0 to 32,
a2=0 to 32,
with the proviso that
a+a1+a2>=2,
b=1 to 600,
b1=0 to 80,
b2=0 to 50,
c=0
c1=0
c2=0
d=0
with the proviso that
a2+b2+c2>=1
and
R¹=independently of one another, identical or different linear or branched, optionally aromatic, hydrocarbon radicals having 1 to 6 carbon atoms;
R²=independently of one another, identical or different linear or branched, optionally aromatic, hydrocarbon radicals 7 to 22, carbon atoms;
R³=independently of one another identical or different organic radicals which have a (meth)acrylate group of the formula (I)
—O—C(O)—CR═CH₂ formula (I)
where R=—H or —CH₃;
R⁵=independently of one another identical or different radicals R¹, R² or R³.

10. The process of claim 8, wherein a silicone of general formula (II) is used, wherein:
a+a1+a2=2 and c+c1+c2+d=0 or
a2=2 and c+c1+c2+d=0.

11. The process of claim 8, wherein a silicone of general formula (II) is used and wherein:
a2=2, b=20 to 400, b2=1 to 20, R¹=CH₃, R³=—C₆H₁₂—O—C(O)—C(CH₃)═CH₂, and
a=a1=b1=c=c1=c2=d=0;
a2=2, b=20 to 400, R¹=CH₃, R³=(CH₂)₃—O—CH₂—C(CH₂—O—C(O)—C(CH₃)═CH₂)₂—CH₂—CH₃, and a=a1=b1=b2=c=c1=c2=d=0;
a2=2, b=20 to 400, b1=1 to 20, R¹=CH₃, R²=C₆ to C₂₄ n-alkyl, R³=(CH₂)₃—O—CH₂—C(CH₂—O—C(O)—C(CH₃)═CH₂)₂—CH₂—CH₃, and a=a1=b2=c=c1=c2 =d=0;
or
a+a2=4, b=20 to 400, b1=1 to 20, c=2, R¹=R⁵=CH₃, R²=C₆ to C₂₄ n-alkyl, R³=(CH₂)₃—O—CH₂—C(CH₂—O—C(O)—C(CH₃)═CH₂)₂—CH₂—CH₃, and a1=b2=c1=c2=d=0.

12. The process of claim 1, wherein the cosmetically suitable carrier medium is a cosmetic oil.

13. The process of claim 1, wherein the organic radical starter is a peroxide derived from an organic acid.

14. The process of claim 13, wherein the organic radical starter is a lauroyl peroxide.

15. A silicone gel, wherein said gel is obtainable by the process of claim 1, and wherein, at a shear rate of 1 l/s and a temperature of 25° C., said silicone gel has a viscosity of less than 150 000 mPas.

16. The silicone of claim 15, wherein, at a shear rate of 1 l/s and a temperature of 25° C., said silicone gel has a viscosity of less than 50 000 mPas.

17. The silicone gel of claim 15, wherein, at a shear rate of 1 l/s and a temperature of 25° C., said silicone gel has a viscosity of less than 15 000 mPas.

18. A cosmetic formulation comprising at least one silicone gel of claim 15.

19. The cosmetic formulation of claim 18, wherein said cosmetic formulation comprises 1% by weight to 40% by weight, of the silicone gel.

20. The cosmetic formulation of claim 18, wherein said cosmetic formulation comprises 3% by weight to 24% by weight, of the silicone gel.
